(19) United States
(12) Patent Application Publication
(10) Pub. No.: US 2006/0189679 A1
(43) Pub. Date: Aug. 24, 2006

(54) C10 cyclopropyl ester substituted taxane compositions

(75) Inventors: Robert A. Holton, Tallahassee, FL (US); Ross E. Longley, Tallahassee, FL (US)

Correspondence Address: SENNIGER POWERS, ONE METROPOLITAN SQUARE, 16TH FLOOR, ST LOUIS, MO, 63102, US

(73) Assignee: Florida State University Research Foundation, Inc., Tallahassee, FL (US)

(21) Appl. No.: 11/352,940

(22) Filed: Feb. 13, 2006

Related U.S. Application Data
(60) Provisional application No. 60/652,834, filed on Feb. 14, 2005.

(51) Int. Cl. A61K 31/337; A61K 31/34

(52) U.S. Cl. 514/449

(57) ABSTRACT

Compositions comprising a taxane having a cyclopropyl ester substituent at C10, a keto substituent at C9, a hydroxy substituent at C7, a 2-furyl substituent at C3′ and an isobutoxycarbamate substituent at C3′.

### BACKGROUND OF THE INVENTION

The present invention is directed to compositions of a C10 cyclopropyl ester substituted taxane having utility as an antitumor agent.

The taxane family of terpenes, of which baccatin III and taxol, also commonly referred to as paclitaxel, are members, has been the subject of considerable interest in both the biological and chemical arts. Taxol (paclitaxel) itself is employed as a cancer chemotherapeutic agent and possesses a broad range of tumor-inhibiting activity. Taxol has a 2′R, 3′S configuration and the following structural formula: 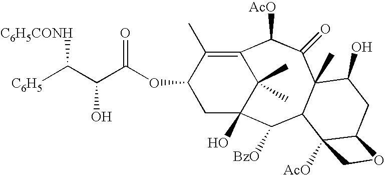
wherein Ac is acetyl and Bz is benzoyl.

Colin et al. reported in U.S. Pat. No. 4,814,470 that certain paclitaxel analogs have an activity significantly greater than that of taxol. One of these analogs, commonly referred to as docetaxel (Taxotere®), has the following structural formula:

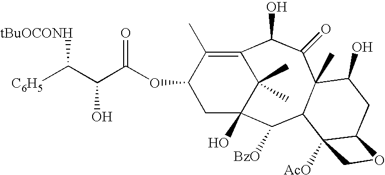

Although taxol and docetaxel are useful chemotherapeutic agents, there are limitations to their effectiveness, including limited efficacy against certain types of cancers and toxicity to subjects when administered at various doses. Further, certain tumors have shown resistance to taxol and/or docetaxel. Accordingly, a need remains for additional chemotherapeutic agents with less toxicity and improved efficacy with respect to taxol and/or docetaxel resistant and non-resistant tumors.

### SUMMARY OF THE INVENTION

Among the various aspects of the present invention, therefore, is the provision of a taxane which compares favorably to taxol and docetaxel with respect to toxicity and to efficacy as an anti-tumor agent, but is also effective with respect to taxol and/or docetaxel resistant tumors. In general, this taxane possesses a cyclopropyl ester substituent at C10, a keto substituent at C9, a hydroxy substituent at C7, a 2-furyl substituent at C3′ and an isobutoxycarbamate substituent at C3′.

Briefly, therefore, the present invention is directed to compositions comprising a taxane effective with respect to taxol and/or docetaxel resistant tumors and a pharmaceutically acceptable carrier and to methods of treatment and administration.

Other objects and features of this invention will be in part apparent and in part pointed out hereinafter.

### BRIEF DESCRIPTION OF THE DRAWINGS

FIG. 1 depicts photographs of A549 human lung cells (control—no treatment).

FIG. 2 depicts photographs of A549 human lung cell treated with compound 3102.

FIG. 3 depicts median tumor growth curves for mice treated with compound 3102 vs. control in the HT-29 colon tumor (e52) study (IV, single dose).

FIG. 4 depicts median tumor growth curves for mice treated with compound 3102 vs. control in the HT-29 colon tumor (e51) study (IV, multi-dose (Q4Dx4)).

FIG. 5 depicts median tumor growth curves for mice treated with compound 3102 vs. control in the HT-29 colon tumor (e60) study (oral, single dose).

FIG. 6 depicts median tumor growth curves for mice treated with compound 3102 vs. control in the HT-29 colon tumor (e76) study (oral, multi-dose (Q4Dx4)).

FIG. 7 depicts median tumor growth curves for mice treated with compound 3102 vs. control in the HT-29 colon tumor (e103) study (oral, single dose).

FIG. 8 depicts median tumor growth curves for mice treated with compound 3102 vs. control in the HT-29 colon tumor (e79) study (oral, multi-dose (Q4Dx4)).

FIG. 9 depicts median tumor growth curves for mice treated with compound 3102 vs. control in the HT-29 colon tumor (e80) study (oral, multi-dose (Q7Dx3)).

FIG. 10 depicts median tumor growth curves for mice treated with compound 3102 vs. paclitaxel and docetaxel in the HT-29 colon tumor (e105) study (oral, multi-dose (Q4Dx4)).

FIG. 11 depicts median tumor growth curves for mice treated with compound 3102 vs. paclitaxel and docetaxel in the HT-29 colon tumor (e105) study (oral, multi-dose (Q7Dx3)).

FIG. 12 depicts median tumor growth curves for mice treated with compound 3102 vs. control in the Panc-1 pancreatic tumor (e59) study (IV, single dose).

FIG. 13 depicts median tumor growth curves for mice treated with compound 3102 vs. paclitaxel in the Panc-1 pancreatic tumor (e57) study (IV, multi-dose (QODx5)).

FIG. 14 depicts median tumor growth curves for mice treated with compound 3102 vs. docetaxel in the Panc-1 pancreatic tumor (e92) study (IV, multi-dose).

FIG. 15 depicts median tumor growth curves for mice treated with compound 3102 vs. control in the Panc-1 pancreatic tumor (e64) study (oral, single dose).

FIG. 16 depicts median tumor growth curves for mice treated with compound 3102 vs. control in the Panc-1 pancreatic tumor (e93) study (oral, single dose).

FIG. 17 depicts median tumor growth curves for mice treated with compound 3102 vs. control in the Panc-1 pancreatic tumor (e79) study (oral, multi-dose, Q4Dx4).

FIG. 18 depicts median tumor growth curves for mice treated with compound 3102 vs. control in the Panc-1 pancreatic tumor (e87) study (oral, multi-dose Q4Dx4).

FIG. 19 depicts median tumor growth curves for mice treated with compound 3102 vs. paclitaxel and docetaxel in the Panc-1 pancreatic tumor (e95) study (oral, multi-dose (Q4Dx4)).

FIG. 20 depicts median tumor growth curves for mice treated with compound 3102 vs. paclitaxel and docetaxel in the Panc-1 pancreatic tumor (e95) study (oral, multi-dose (Q7Dx3)).

FIG. 21 depicts median tumor growth curves for mice treated with compound 3102 vs. paclitaxel and docetaxel in the DLD-1 colon tumor study (oral, multi-dose (Q4Dx4)).

FIG. 22 depicts median tumor growth curves for mice treated with compound 3102 vs. paclitaxel and docetaxel in the SW480 colon tumor study (oral and IV, multi-dose (Q4Dx4)).

FIG. 23 depicts median tumor growth curves for mice treated with compound 3102 vs. paclitaxel and docetaxel in the 786-0 renal tumor study (oral, multi-dose (Q4Dx4)).

FIG. 24 depicts median tumor growth curves for mice treated with compound 3102 vs. docetaxel in the MSTO-211H mesothelioma study (oral, multi-dose (Q4Dx4)).

FIG. 25 depicts body weight changes for mice treated with compound 3102 vs. docetaxel in the MSTO-211H mesothelioma study (oral, multi-dose (Q4Dx4)).

### DETAILED DESCRIPTION OF THE PREFERRED EMBODIMENTS

The taxane of the present invention, compound 3102, has the following chemical structure:

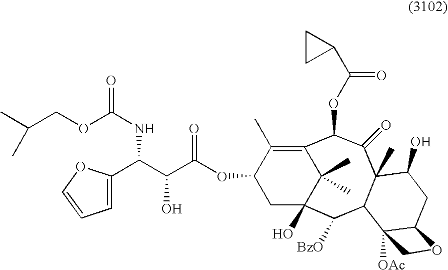

Compound 3102 is active against cancers both in vitro and in vivo in a manner superior to conventionally used taxanes with respect to certain tumor types, including paclitaxel and/or docetaxel sensitive and resistant tumor lines. Whether or not used in combination with other agents, pharmaceutical compositions comprising compound 3102 may be used to treat those cancers indicated for treatment with Taxol® and/or Taxotere®. Without being limiting, pharmaceutical compositions comprising compound 3102 may be used, either solely or in combination, to treat breast cancer, non-small cell lung cancer, prostate cancer, ovarian cancer, and AIDS-related Kaposi's sarcoma. The compound is reasonably well tolerated whether administered orally or intravenously and can be effective as a single or multiple dose with improved toxicity profiles.

It is believed that the mechanism of action of compound 3102 includes microtubule polymerization, resulting in a block in the G₂/M phase of the cell cycle and programmed cell death, known as apoptosis. This compound is highly efficacious in a number of human tumor nude mouse xenograft models, including those which are refractory/resistant to paclitaxel and Taxotere® (docetaxel). Compound 3102 can be effectively dosed via the intravenous and oral routes on a single or multidose schedule. In the majority of xenograft models tested, compound 3102 shows superior efficacy to paclitaxel and Taxotere® when administered as an oral dose and on a multi-dose schedule, either every 4 days or every 7 days. Compound 3102 shows a wide therapeutic index in these mouse xenograft models. Doses well below the maximum tolerated dose, as indicated by body weight loss, still maintain efficacy. The compound displays superior bioavailability orally as demonstrated by efficacy observed in xenograft models and in a favorable toxicity profile when dosed both orally and IV in Sprague-Dawley rats. The superior efficacy and wide therapeutic index in multiple dosing regimens suggests an opportunity for increased dose intensity in the clinic particularly when dosed weekly in human studies.

Compound 3102 may be obtained by treatment of a β-lactam with an alkoxide having the taxane tetracyclic nucleus and a C13 metallic oxide substituent to form compounds having a β-amido ester substituent at C13 (as described more fully in Holton U.S. Pat. No. 5,466,834), followed by removal of the hydroxy protecting groups. The β-lactam has the following structural formula (1): 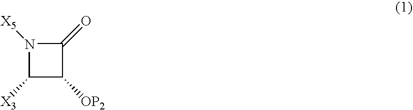
wherein P₂ is a hydroxy protecting group, X₃ is 2-furyl, and X₅ is isobutoxycarbonyl and the alkoxide has the structural formula (2): 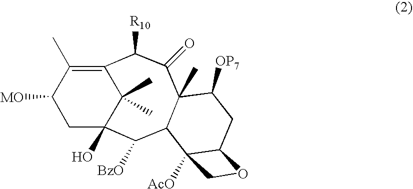
wherein M is a metal or ammonium, P₇ is a hydroxy protecting group and R₁₀ is cyclopropylcarbonyloxy.

The alkoxide of structural formula (2) may be prepared from 10-deacetylbaccatin III (or a derivative thereof) by selective protection of the C7 hydroxyl group and then esterification of the C10 hydroxyl group followed by treatment with a metallic amide. In one embodiment of the present invention, the C7 hydroxyl group of 10-deacetylbaccatin III is selectively protected with a silyl group as described, for example, by Denis, et. al. (J. Am. Chem. Soc., 1988, 110, 5917). In general, the silylating agents may be used either alone or in combination with a catalytic amount of a base such as an alkali metal base.

Alternatively, the C10 hydroxyl group of a taxane can be selectively acylated in the absence of a base, as described, for example in Holton et al., PCT Patent Application WO 99/09021. Acylating agents which may be used for the selective acylation of the C10 hydroxyl group of a taxane include substituted or unsubstituted alkyl or aryl anhydrides. While the acylation of the C10 hydroxy group of the taxane will proceed at an adequate rate for many acylating agents, it has been discovered that the reaction rate may be increased by including a Lewis acid in the reaction mixture. Preferred Lewis acids include zinc chloride, stannic chloride, cerium trichloride, cuprous chloride, lanthanum trichloride, dysprosium trichloride, and ytterbium trichloride. Zinc chloride or cerium trichloride is particularly preferred when the acylating agent is an anhydride.

Processes for the preparation and resolution of the β-lactam starting material are generally well known in the art. For example, the β-lactam may be prepared as described in Holton, U.S. Pat. No. 5,430,160 (col. 9, lines 2-50) or Holton, U.S. Pat. No. 6,649,632 (col. 7, line 45—col. 8, line 60), which are both hereby incorporated by this reference in their entirety. The resulting enatiomeric mixtures of β-lactams may be resolved by a stereoselective hydrolysis using a lipase or enzyme as described, for example, in Patel, U.S. Pat. No. 5,879,929 (col. 16, lines 1—col. 18, line 27) or Patel, U.S. Pat. No. 5,567,614 or a liver homogenate as described, for example, in Holton, U.S. Pat. No. 6,548,293 (col. 3, lines 30-61). By way of example, U.S. Pat. No. 6,649,632 discloses the preparation of a β-lactam having a furyl substituent at the C4 position of the β-lactam.

The taxane of the instant invention is useful for inhibiting tumor growth in mammals including humans and is preferably administered in the form of a pharmaceutical composition comprising an effective antitumor amount of the compound of the instant invention in combination with at least one pharmaceutically or pharmacologically acceptable carrier. The carrier, also known in the art as an excepient, vehicle, auxiliary, adjuvant, or diluent, is any substance which is pharmaceutically inert, confers a suitable consistency or form to the composition, and does not diminish the therapeutic efficacy of the antitumor compounds. The carrier is “pharmaceutically or pharmacologically acceptable” if it does not produce an adverse, allergic or other untoward reaction when administered to a mammal or human, as appropriate.

The pharmaceutical compositions containing the antitumor compound of the present invention may be formulated in any conventional manner. Proper formulation is dependent upon the route of administration chosen. The compositions of the invention can be formulated for any route of administration so long as the target tissue is available via that route. Suitable routes of administration include, but are not limited to, oral, parenteral (e.g., intravenous, intraarterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration.

Pharmaceutically acceptable carriers for use in the compositions of the present invention are well known to those of ordinary skill in the art and are selected based upon a number of factors: the particular antitumor compound used, and its concentration, stability and intended bioavailability; the disease, disorder or condition being treated with the composition; the subject, its age, size and general condition; and the route of administration. Suitable carriers are readily determined by one of ordinary skill in the art (see, for example, J. G. Nairn, in: Remington's Pharmaceutical Science (A. Gennaro, ed.), Mack Publishing Co., Easton, Pa., (1985), pp. 1492-1517, the contents of which are incorporated herein by reference).

The compositions are preferably formulated as tablets, dispersible powders, pills, capsules, gelcaps, caplets, gels, liposomes, granules, solutions, suspensions, emulsions, syrups, elixirs, troches, dragees, lozenges, or any other dosage form which can be administered orally. Techniques and compositions for making oral dosage forms useful in the present invention are described in the following references: 7 Modern Pharmaceutics, Chapters 9 and 10 (Banker & Rhodes, Editors, 1979); Lieberman et al., Pharmaceutical Dosage Forms: Tablets (1981); and Ansel, Introduction to Pharmaceutical Dosage Forms 2nd Edition (1976).

The compositions of the invention for oral administration comprise an effective antitumor amount of the compound of the invention in a pharmaceutically acceptable carrier. Suitable carriers for solid dosage forms include sugars, starches, and other conventional substances including lactose, talc, sucrose, gelatin, carboxymethylcellulose, agar, mannitol, sorbitol, calcium phosphate, calcium carbonate, sodium carbonate, kaolin, alginic acid, acacia, corn starch, potato starch, sodium saccharin, magnesium carbonate, tragacanth, microcrystalline cellulose, colloidal silicon dioxide, croscarmellose sodium, talc, magnesium stearate, and stearic acid. Further, such solid dosage forms may be uncoated or may be coated by known techniques; e.g., to delay disintegration and absorption.

The antitumor compound of the present invention may also be preferably formulated for parenteral administration, e.g., formulated for injection via intravenous, intraarterial, subcutaneous, rectal, subcutaneous, intramuscular, intraorbital, intracapsular, intraspinal, intraperitoneal, or intrasternal routes. The compositions of the invention for parenteral administration comprise an effective antitumor amount of the antitumor compound in a pharmaceutically acceptable carrier. Dosage forms suitable for parenteral administration include solutions, suspensions, dispersions, emulsions or any other dosage form which can be administered parenterally. Techniques and compositions for making parenteral dosage forms are known in the art.

Suitable carriers used in formulating liquid dosage forms for oral or parenteral administration include nonaqueous, pharmaceutically-acceptable polar solvents such as oils, alcohols, amides, esters, ethers, ketones, hydrocarbons and mixtures thereof, as well as water, saline solutions, dextrose solutions (e.g., DW5), electrolyte solutions, or any other aqueous, pharmaceutically acceptable liquid.

Suitable nonaqueous, pharmaceutically-acceptable polar solvents include, but are not limited to, alcohols (e.g., α-glycerol formal, β-glycerol formal, 1,3-butyleneglycol, aliphatic or aromatic alcohols having 2-30 carbon atoms such as methanol, ethanol, propanol, isopropanol, butanol, t-butanol, hexanol, octanol, amylene hydrate, benzyl alcohol, glycerin (glycerol), glycol, hexylene glycol, tetrahydrofurfuryl alcohol, lauryl alcohol, cetyl alcohol, or stearyl alcohol, fatty acid esters of fatty alcohols such as polyalkylene glycols (e.g., polypropylene glycol, polyethylene glycol), sorbitan, sucrose and cholesterol); amides (e.g., dimethylacetamide (DMA), benzyl benzoate DMA, dimethylformamide, N-(β-hydroxyethyl)-lactamide, N,N-dimethylacetamide amides, 2-pyrrolidinone, 1-methyl-2-pyrrolidinone, or polyvinylpyrrolidone); esters (e.g., 1-methyl-2-pyrrolidinone, 2-pyrrolidinone, acetate esters such as monoacetin, diacetin, and triacetin, aliphatic or aromatic esters such as ethyl caprylate or octanoate, alkyl oleate, benzyl benzoate, benzyl acetate, dimethylsulfoxide (DMSO), esters of glycerin such as mono, di, or tri-glyceryl citrates or tartrates, ethyl benzoate, ethyl acetate, ethyl carbonate, ethyl lactate, ethyl oleate, fatty acid esters of sorbitan, fatty acid derived PEG esters, glyceryl monostearate, glyceride esters such as mono, di, or tri-glycerides, fatty acid esters such as isopropyl myristrate, fatty acid derived PEG esters such as PEG-hydroxyoleate and PEG-hydroxystearate, N-methylpyrrolidinone, pluronic 60, polyoxyethylene sorbitol oleic polyesters such as poly(ethoxylated)₃₀₋₆₀ sorbitol poly(oleate)₂₋₄, poly(oxyethylene)₁₅₋₂₀ monooleate, poly(oxyethylene)₁₅₋₂₀ mono 12-hydroxystearate, and poly(oxyethylene)₁₅₋₂₀ mono ricinoleate, polyoxyethylene sorbitan esters such as polyoxyethylene-sorbitan monooleate, polyoxyethylene-sorbitan monopalmitate, polyoxyethylene-sorbitan monolaurate, polyoxyethylene-sorbitan monostearate, and Polysorbate® 20, 40, 60 or 80 from ICI Americas, Wilmington, Del., polyvinylpyrrolidone, alkyleneoxy modified fatty acid esters such as polyoxyl 40 hydrogenated castor oil and polyoxyethylated castor oils (e.g., Cremophor® EL solution or Cremophor® RH 40 solution), saccharide fatty acid esters (i.e., the condensation product of a monosaccharide (e.g., pentoses such as ribose, ribulose, arabinose, xylose, lyxose and xylulose, hexoses such as glucose, fructose, galactose, mannose and sorbose, trioses, tetroses, heptoses, and octoses), disaccharide (e.g., sucrose, maltose, lactose and trehalose) or oligosaccharide or mixture thereof with a C₄-C₂₂ fatty acid(s)(e.g., saturated fatty acids such as caprylic acid, capric acid, lauric acid, myristic acid, palmitic acid and stearic acid, and unsaturated fatty acids such as palmitoleic acid, oleic acid, elaidic acid, erucic acid and linoleic acid)), or steroidal esters); alkyl, aryl, or cyclic ethers having 2-30 carbon atoms (e.g., diethyl ether, tetrahydrofuran, dimethyl isosorbide, diethylene glycol monoethyl ether); glycofurol (tetrahydrofurfuryl alcohol polyethylene glycol ether); ketones having 3-30 carbon atoms (e.g., acetone, methyl ethyl ketone, methyl isobutyl ketone); aliphatic, cycloaliphatic or aromatic hydrocarbons having 4-30 carbon atoms (e.g., benzene, cyclohexane, dichloromethane, dioxolanes, hexane, n-decane, n-dodecane, n-hexane, sulfolane, tetramethylenesulfon, tetramethylenesulfoxide, toluene, dimethylsulfoxide (DMSO), or tetramethylenesulfoxide); oils of mineral, vegetable, animal, essential or synthetic origin (e.g., mineral oils such as aliphatic or wax-based hydrocarbons, aromatic hydrocarbons, mixed aliphatic and aromatic based hydrocarbons, and refined paraffin oil, vegetable oils such as linseed, tung, safflower, soybean, castor, cottonseed, groundnut, rapeseed, coconut, palm, olive, corn, corn germ, sesame, persic and peanut oil and glycerides such as mono-, di- or triglycerides, animal oils such as fish, marine, sperm, cod-liver, haliver, squalene, squalane, and shark liver oil, oleic oils, and polyoxyethylated castor oil); alkyl or aryl halides having 1-30 carbon atoms and optionally more than one halogen substituent; methylene chloride; monoethanolamine; petroleum benzin; trolamine; omega-3 polyunsaturated fatty acids (e.g., alpha-linolenic acid, eicosapentaenoic acid, docosapentaenoic acid, or docosahexaenoic acid); polyglycol ester of 12-hydroxystearic acid and polyethylene glycol (Solutol® HS-15, from BASF, Ludwigshafen, Germany); polyoxyethylene glycerol; sodium laurate; sodium oleate; or sorbitan monooleate.

Other pharmaceutically acceptable solvents for use in the invention are well known to those of ordinary skill in the art, and are identified in The Chemotherapy Source Book (Williams & Wilkens Publishing), The Handbook of Pharmaceutical Excipients, (American Pharmaceutical Association, Washington, D.C., and The Pharmaceutical Society of Great Britain, London, England, 1968), Modern Pharmaceutics, (G. Banker et al., eds., 3d ed.)(Marcel Dekker, Inc., New York, N.Y., 1995), The Pharmacological Basis of Therapeutics, (Goodman & Gilman, McGraw Hill Publishing), Pharmaceutical Dosage Forms, (H. Lieberman et al., eds.,)(Marcel Dekker, Inc., New York, N.Y., 1980), Remington's Pharmaceutical Sciences (A. Gennaro, ed., 19th ed.)(Mack Publishing, Easton, Pa., 1995), The United States Pharmacopeia 24, The National Formulary 19, (National Publishing, Philadelphia, Pa., 2000), A. J. Spiegel et al., and Use of Nonaqueous Solvents in Parenteral Products, Journal of Pharmaceutical Sciences, Vol. 52, No. 10, pp. 917-927 (1963).

Preferred solvents include those known to stabilize the antitumor compound, such as oils rich in triglycerides, for example, safflower oil, soybean oil or mixtures thereof, and alkyleneoxy modified fatty acid esters such as polyoxyl 40 hydrogenated castor oil and polyoxyethylated castor oils (e.g., Cremophor® EL solution or Cremophor® RH 40 solution). Commercially available triglyceride-rich oils include Intralipid® emulsified soybean oil (Kabi-Pharmacia Inc., Stockholm, Sweden), Nutralipid® emulsion (McGaw, Irvine, Calif.), Liposyn® II 20% emulsion (a 20% fat emulsion solution containing 100 mg safflower oil, 100 mg soybean oil, 12 mg egg phosphatides, and 25 mg glycerin per ml of solution; Abbott Laboratories, Chicago, Ill.), Liposyn® III 20% emulsion (a 20% fat emulsion solution containing 100 mg safflower oil, 100 mg soybean oil, 12 mg egg phosphatides, and 25 mg glycerin per ml of solution; Abbott Laboratories, Chicago, Ill.), natural or synthetic glycerol derivatives containing the docosahexaenoyl group at levels between 25% and 100% by weight based on the total fatty acid content (Dhasco® (from Martek Biosciences Corp., Columbia, Md.), DHA Maguro® (from Daito Enterprises, Los Angeles, Calif.), Soyacal®, and Travemulsion®. Ethanol is a preferred solvent for use in dissolving the antitumor compound to form solutions, emulsions, and the like.

Additional minor components can be included in the compositions of the invention for a variety of purposes well known in the pharmaceutical industry. These components will for the most part impart properties which enhance retention of the antitumor compound at the site of administration, protect the stability of the composition, control the pH, facilitate processing of the antitumor compound into pharmaceutical formulations, and the like. Preferably, each of these components is individually present in less than about 15 weight % of the total composition, more preferably less than about 5 weight %, and most preferably less than about 0.5 weight % of the total composition. Some components, such as fillers or diluents, can constitute up to 90 wt. % of the total composition, as is well known in the formulation art. Such additives include cryoprotective agents for preventing reprecipitation of the taxane, surface active, wetting or emulsifying agents (e.g., lecithin, polysorbate-80, pluronic 60, polyoxyethylene stearate, and polyoxyethylated castor oils), preservatives (e.g., ethyl-p-hydroxybenzoate), microbial preservatives (e.g., benzyl alcohol, phenol, m-cresol, chlorobutanol, sorbic acid, thimerosal and paraben), agents for adjusting pH or buffering agents (e.g., acids, bases, sodium acetate, sorbitan monolaurate), agents for adjusting osmolarity (e.g., glycerin), thickeners (e.g., aluminum monostearate, stearic acid, cetyl alcohol, stearyl alcohol, guar gum, methyl cellulose, hydroxypropylcellulose, tristearin, cetyl wax esters, polyethylene glycol), colorants, dyes, flow aids, non-volatile silicones (e.g., cyclomethicone), clays (e.g., bentonites), adhesives, bulking agents, flavorings, sweeteners, adsorbents, fillers (e.g., sugars such as lactose, sucrose, mannitol, or sorbitol, cellulose, or calcium phosphate), diluents (e.g., water, saline, electrolyte solutions), binders (e.g., starches such as maize starch, wheat starch, rice starch, or potato starch, gelatin, gum tragacanth, methyl cellulose, hydroxypropyl methylcellulose, sodium carboxymethyl cellulose, polyvinylpyrrolidone, sugars, polymers, acacia), disintegrating agents (e.g., starches such as maize starch, wheat starch, rice starch, potato starch, or carboxymethyl starch, cross-linked polyvinyl pyrrolidone, agar, alginic acid or a salt thereof such as sodium alginate, croscarmellose sodium or crospovidone), lubricants (e.g., silica, talc, stearic acid or salts thereof such as magnesium stearate, or polyethylene glycol), coating agents (e.g., concentrated sugar solutions including gum arabic, talc, polyvinyl pyrrolidone, carbopol gel, polyethylene glycol, or titanium dioxide), and antioxidants (e.g., sodium metabisulfite, sodium bisulfite, sodium sulfite, dextrose, phenols, and thiophenols).

Dosage form administration by these routes may be continuous or intermittent, depending, for example, upon the patient's physiological condition, whether the purpose of the administration is therapeutic or prophylactic, and other factors known to and assessable by a skilled practitioner.

Dosage and regimens for the administration of the pharmaceutical compositions of the invention can be readily determined by those with ordinary skill in treating cancer. It is understood that the dosage of the antitumor compounds will be dependent upon the age, sex, health, and weight of the recipient, kind of concurrent treatment, if any, frequency of treatment, and the nature of the effect desired. For any mode of administration, the actual amount of antitumor compound delivered, as well as the dosing schedule necessary to achieve the advantageous effects described herein, will also depend, in part, on such factors as the bioavailability of the antitumor compound, the disorder being treated, the desired therapeutic dose, and other factors that will be apparent to those of skill in the art. The dose administered to an animal, particularly a human, in the context of the present invention should be sufficient to effect the desired therapeutic response in the animal over a reasonable period of time. Preferably, an effective amount of the antitumor compound, whether administered orally or by another route, is any amount which would result in a desired therapeutic response when administered by that route. Preferably, the compositions for oral administration are prepared in such a way that a single dose in one or more oral preparations contains at least 20 mg of the antitumor compound per m² of patient body surface area, or at least 50, 100, 150, 200, 300, 400, or 500 mg of the antitumor compound per m² Of patient body surface area, wherein the average body surface area for a human is 1.8 m². Preferably, a single dose of a composition for oral administration contains from about 20 to about 600 mg of the antitumor compound per m² of patient body surface area, more preferably from about 25 to about 400 mg/m²′ even more preferably, from about 40 to about 300 mg/m², and even more preferably from about 50 to about 200 mg/m². Preferably, the compositions for parenteral administration are prepared in such a way that a single dose contains at least 20 mg of the antitumor compound per m² Of patient body surface area, or at least 40, 50, 100, 150, 200, 300, 400, or 500 mg of the antitumor compound per m² of patient body surface area. Preferably, a single dose in one or more parenteral preparations contains from about 20 to about 500 mg of the antitumor compound per m² Of patient body surface area, more preferably from about 40 to about 400 mg/m², and even more preferably, from about 60 to about 350 mg/m². However, the dosage may vary depending on the dosing schedule which can be adjusted as necessary to achieve the desired therapeutic effect. It should be noted that the ranges of effective doses provided herein are not intended to limit the invention and represent preferred dose ranges. The most preferred dosage will be tailored to the individual subject, as is understood and determinable by one of ordinary skill in the art without undue experimentation.

The concentration of the antitumor compound in a liquid pharmaceutical composition is preferably between about 0.01 mg and about 10 mg/mL of the composition, more preferably between about 0.1 mg and about 7 mg/mL, even more preferably between about 0.5 mg and about 5 mg/mL, and most preferably between about 1.5 mg and about 4 mg per ml. In one embodiment, the concentration of 3102 in this formulation is 2 to 4 mg/mL. Relatively low concentrations are generally preferred because the antitumor compound is most soluble in the solution at low concentrations. The concentration of the antitumor compound in a solid pharmaceutical composition for oral administration is preferably between about 5 weight % and about 50 weight %, based on the total weight of the composition, more preferably between about 8 weight % and about 40 weight %, and most preferably between about 10 weight % and about 30 weight %.

In one embodiment, solutions for oral administration are prepared by dissolving an antitumor compound in any pharmaceutically acceptable solvent capable of dissolving the compound (e.g., ethanol or polyethylene glycol) to form a solution. An appropriate volume of a carrier which is a surfactant, such as Cremophor® EL solution, polysorbate 80, Solutol HS15, or Vitamin E TPGS, is added to the solution while stirring to form a pharmaceutically acceptable solution for oral administration to a patient. For example, the resulting compositions may contain up to about 15% ethanol and/or up to about 15% surfactant, more typically, the concentrations will be about 7.5-15% by volume ethanol with an equal volume of surfactant and distilled water in the range of 75-90% by volume. For taste purposes, a fraction of the distilled water can be replaced by a diluted cherry or raspberry syrup, preferably, about 10-30% syrup with the remainder water. In one embodiment, the concentration of 3102 in this formulation is 2 to 4 mg/mL. If desired, such solutions can be formulated to contain a minimal amount of, or to be free of, ethanol, which is known in the art to cause adverse physiological effects when administered at certain concentrations in oral formulations. In a preferred embodiment, the solution comprises about 10% ethanol, about 10% surfactant selected from polysorbate 80 (e.g., Tween 80®), polyethoxylated caster oils (e.g., Cremophor®), and mixtures thereof, and about 80% distilled water.

In another embodiment, powders or tablets for oral administration are prepared by dissolving an antitumor compound in any pharmaceutically acceptable solvent capable of dissolving the compound (e.g., ethanol or polyethylene glycol) to form a solution. The solvent can optionally be capable of evaporating when the solution is dried under vacuum. An additional carrier can be added to the solution prior to drying, such as Cremophor® EL solution. The resulting solution is dried under vacuum to form a glass. The glass is then mixed with a binder to form a powder. The powder can be mixed with fillers or other conventional tabletting agents and processed to form a tablet for oral administration to a patient. The powder can also be added to any liquid carrier as described above to form a solution, emulsion, suspension or the like for oral administration.

Emulsions for parenteral administration can be prepared by dissolving an antitumor compound in any pharmaceutically acceptable solvent capable of dissolving the compound (e.g., ethanol or polyethylene glycol) to form a solution. An appropriate volume of a carrier which is an emulsion, such as Liposyn® II, Liposyn® III, or Intralipid® emulsion, is added to the solution while stirring to form a pharmaceutically acceptable emulsion for parenteral administration to a patient. For example, the resulting composition may contain up to about 10% ethanol and/or more than about 90% carrier, more typically, the concentration will be about 5-10% by volume ethanol and about 90-95% by volume carrier. In one embodiment, the concentration of 3102 in the dosing solution is about 1-2 mg/mL. If desired, such emulsions can be formulated to contain a minimal amount of, or to be free of, ethanol or Cremophor® solution, which are known in the art to cause adverse physiological effects when administered at certain concentrations in parenteral formulations. In a preferred embodiment, the emulsion comprises about 5% ethanol and about 95% carrier (e.g., Intralipid 20%, Liposyn II 20%, or a mixture thereof). In this preferred embodiment, the emulsion is free of agents which are known to cause adverse physiological effects, such as polyethoxylated caster oils (e.g., Cremophor®) and polysorbate 80 (e.g., Tween 80®).

Solutions for parenteral administration can be prepared by dissolving an antitumor compound in any pharmaceutically acceptable solvent capable of dissolving the compound (e.g., ethanol or polyethylene glycol) to form a solution. An appropriate volume of a carrier which is a surfactant, such as Cremophor® solution, polysorbate 80, or Solutol HS15, is added to the solution while stirring to form a pharmaceutically acceptable solution for parenteral administration to a patient. For example, the resulting composition may contain up to about 10% ethanol and/or up to about 10% surfactant, more typically, the concentration will be about 5-10% by volume ethanol with an equal volume of surfactant and saline in the range of 80-90% by volume. If desired, such solutions can be formulated to contain a minimal amount of, or to be free of, ethanol or Cremophor® solution, which are known in the art to cause adverse physiological effects when administered at certain concentrations in parenteral formulations. In a preferred embodiment, the solution comprises about 5% ethanol, about 5% polysorbate 80 (e.g., Tween 80®) or polyethoxylated caster oils (e.g., Cremophor®), and about 90% saline (0.9% sodium chloride). To minimize or eliminate potential adverse effects (e.g., hypersensitivity reactions), a patient receiving this embodiment is preferably pretreated with dexamethasone, diphenhydramine, or any other agent known in the art to minimize or eliminate these adverse reactions.

Other suitable parenteral formulations include liposomes. Liposomes are generally spherical or spheroidal clusters or aggregates of amphiphatic compounds, including lipid compouds, typically in the form of one or more concentric layers, for example monolayers or bilayers. The liposomes may be formulated from either ionic or nonionic lipids. Liposomes from nonionic lipids are also referred to as niosomes. References for liposomes include: (a) Liposomes Second Edition: A Practical Approach, edited by V. Torchillin and V. Weissig, Oxford University Press, 2003; (b) M. Malmstein, Surfactants and Polymers in Drug Delivery, Marcel Dekker Inc., 2002; and (c) Muller et al., Emulsions and Nanosuspensions for the Formulation of Poorly Soluble Drugs, Medpharm Scientific Publishers, 1998.

If desired, the emulsions or solutions described above for oral or parenteral administration can be packaged in IV bags, vials or other conventional containers in concentrated form and diluted with any pharmaceutically acceptable liquid, such as saline, to form an acceptable taxane concentration prior to use as is known in the art.

The terms “hydroxyl protecting group” and “hydroxy protecting group” as used herein denote a group capable of protecting a free hydroxyl group (“protected hydroxyl”) which, subsequent to the reaction for which protection is employed, may be removed without disturbing the remainder of the molecule. A variety of protecting groups for the hydroxyl group and the synthesis thereof may be found in Protective Groups in Organic Synthesis, 3rd Edition by T. W. Greene and P. G. M. Wuts, John Wiley and Sons, 1999. Exemplary hydroxylprotecting groups include methoxymethyl, 1-ethoxyethyl, benzyloxymethyl, (β-trimethylsilylethoxy)methyl, tetrahydropyranyl, 2,2,2-trichloroethoxycarbonyl, t-butyl(diphenyl)silyl, trialkylsilyl, trichloromethoxycarbonyl and 2,2,2-trichloroethoxymethyl.

As used herein, “Ac” means acetyl; “Bz” means benzoyl; “TES” means triethylsilyl; “TMS” means trimethylsilyl; “LAH” means lithium aluminum hydride; “10-DAB” means 10-desacetylbaccatin III; “THF” means tetrahydrofuran; “DMAP” means 4-dimethylamino pyridine; “LHMDS” means lithium hexamethyldisilazanide; “TESCI” means triethylsilyl chloride; “cPtc-CI” means cyclopentanecarbonyl chloride; “DMF” means N,N-dimethylformamid; “MOP” means 2-methoxypropene; “iProc” means N-isopropoxycarbonyl; “iProc-CI” means isopropyl chloroformate; and “LDA” means lithium diisopropylamide.

The following examples illustrate the invention.

### EXAMPLE 1

### Preparation of Compound 3102

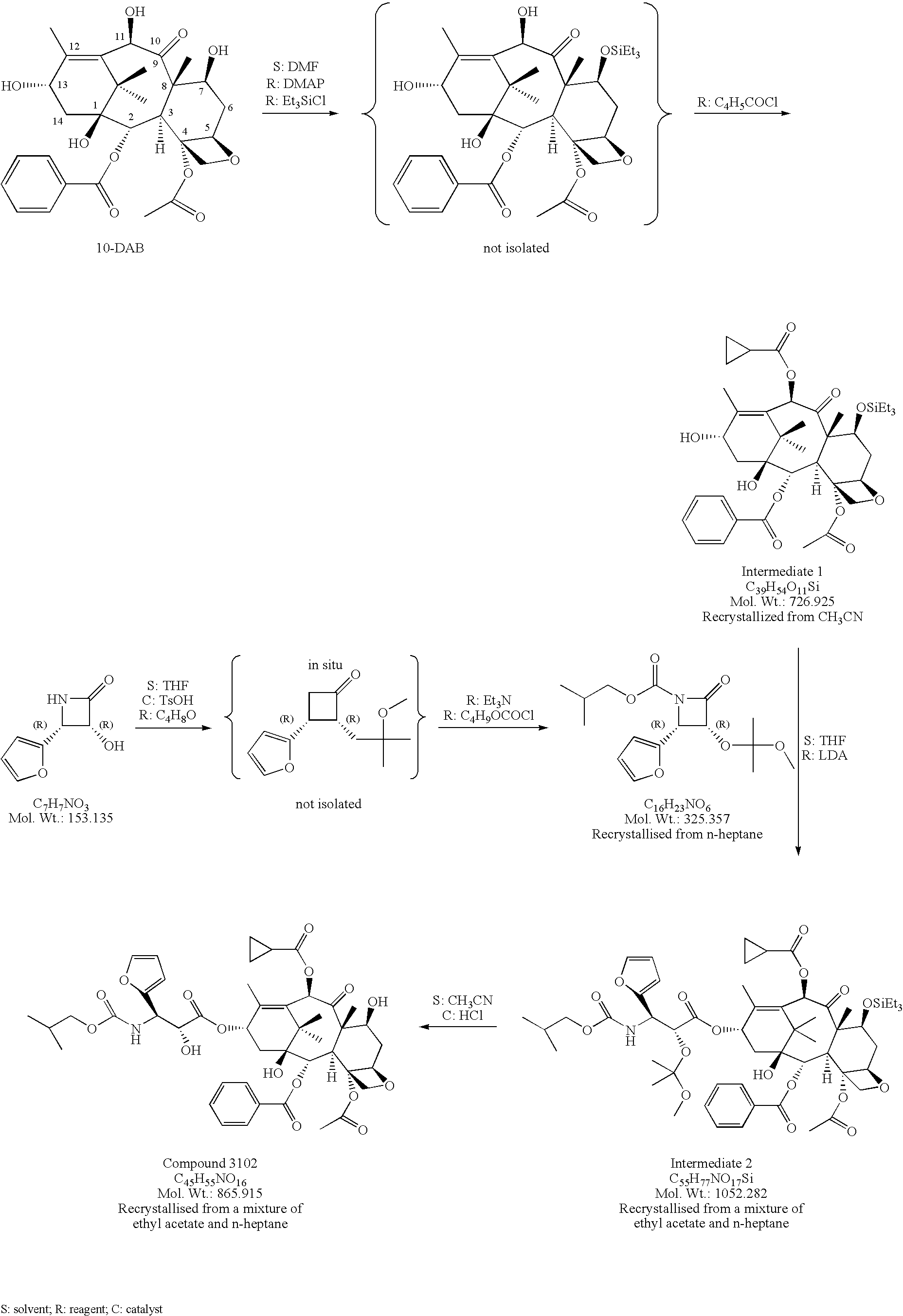

### EXAMPLE 2

### Microtubule Stabilization

Compound 3102 was evaluated for its ability to stabilize microtubules in living tumor cells in vitro, the result of which is cell death and which is ascribed as the mechanism of action for the anticancer drugs paclitaxel and docetaxel.

Briefly, approximately 5,000 A549 human lung cancer cells in complete tissue culture medium (RPMI 1640 medium with 10% fetal calf serum and antibiotics) were added to wells of slide chambers and allowed to grow and attach overnight. Varying dilutions of compound 3102, paclitaxel and docetaxel in dimethyl sulfoxide (DMSO) were prepared from initial 1.0 mM stock solutions and were added to the slide chamber wells and incubated at 37° C. for 24 hours. Slides were fixed with 10% formalin containing 3% glucose for 10 min at room temperature, washed with phosphate buffered solution (PBS) and incubated with 2% triton X-100 in PBS then stained with a 1:1000 dilution of mouse anti-α tubulin for 45 min at 370° C., followed by three washes and stained with fluorescein isothiocyanate (FITC) conjugated, goat anti-mouse antibody and similarly incubated for 45 min at 37° C. Antibody solution was removed, and a propidium iodide/RNAse solution was added and the slides incubated at 37° C. for and additional 20 min. Slides were washed with PBS and distilled water and allowed to air dry. Cover slips were mounted to slides with SlowFade and the slides examined using fluorescence microscopy.

Results: Microtubule Stabilization of HCT116 Tumor Cells.

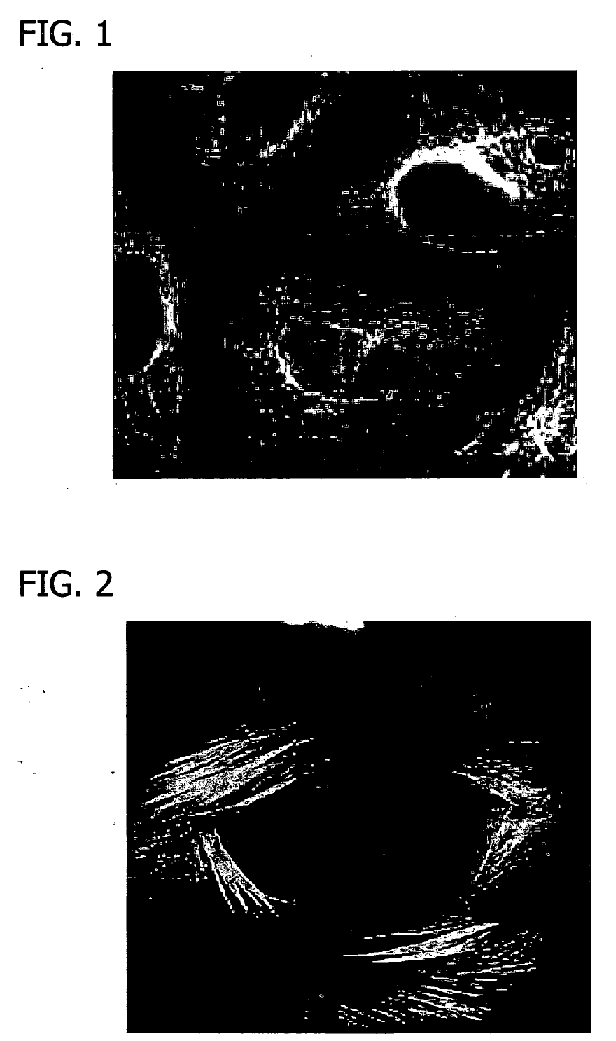

The microtubule matrix of untreated, A549 cells is characterized by a mesh-like network of tubular structures (microtubules) (FIG. 1). A549 cells treated with 100 nM of compound 3102 demonstrated formation of “bundles” of microtubules, some of which run the entire length of the cell (FIG. 2). Nuclei of these cells (ovoid structures in photograph) expressed fragmentation which is indicative of apoptosis. Similar effects on microtubules and nuclei were observed with paclitaxel and docetaxel treated cells. The results show that compound 3102 induces both microtubule bundling and apoptosis in vitro, a mechanism of action which is consistent with that of paclitaxel and docetaxel.

### EXAMPLE 3

### Cell Cycle and Apoptotic Analyses

Studies were initiated to identify the cell cycle phases within the cell cycle by which compound 3102 was exerting its antiproliferative effect against HCT116 cells in comparison to paclitaxel and docetaxel.

HCT116 human colon carcinoma cells were incubated in the presence or absence of (10.0, and 100.0, nM) of compound 3102, paclitaxel or docetaxel for 24 and 48 hr. Cells were harvested, fixed in 75% ethanol overnight at 4° C. and stained with 0.02 mg/ml of propidium iodide (PI) together with 0.1 mg/ml of RNAse A and analyzed on a Coulter ALTRA flow cytometer. DNA histograms were collected from at least 10,000 P.I. stained cells at an emission wavelength of 690 nM. The number of cells in each phase of the cell cycle (G₁, S and G₂/M) was determined and those in the apoptotic phase were measured by determining the percentage of cells in sub G₁ peak.

Results: Effect of Compound 3102 on Cell Cycle and Apoptosis of HCT-116 cells

Increasing concentrations of compound 3102, paclitaxel and docetaxel resulted in decreased percentages of cells in G₁ phase, with a concomitant increase in the percentage of cells in S and G₂/M phases of the cell cycle compared to control (untreated) following 24 hr exposure. Compound 3102 and paclitaxel induced very similar effects on the percentage of cells undergoing apoptosis at 10.0 nM, while docetaxel treated cell populations appeared to be both necrotic and apoptotic at this concentration. These results indicate that the mechanism of action of compound 3102, i.e. blockage of cell proliferation in the G₂/M phase of the cell cycle and the induction of apoptosis is consistent with that of both paclitaxel and docetaxel. The results are summarized in Table 1 below.

TABLE 1
Cell Cycle Effects of Compound 3102 on HCT-116 Cells

| Treatment | Apoptotic | G₁ | S | G₂/M |
|---|---|---|---|---|
| Control | 1.1 | 58.7 | 19.6 | 20.8 |
| 3102 (10 nM) | 24.4 | 20.1 | 23.9 | 27.3 |
| 3102 (100 nM) | 11.7 | 7.0 | 7.1 | 74.0 |
| Paclitaxel (10 nM) | 24.0 | 31.1 | 25.0 | 17.8 |
| Paclitaxel (100 nM) | 15.3 | 7.0 | 9.4 | 67.2 |
| Docetaxel (10 nM) | 44.1 | 10.4 | 25.4 | 17.1 |
| Docetaxel (100 nM) | 14.4 | 4.0 | 11.6 | 68.0 |

### EXAMPLE 4

### Comparison of In Vitro Cytotoxic Activity of Compound 3102 to Taxanes

The in vitro cytotoxic activity of compound 3102 was compared to that of other known taxanes (paclitaxel and docetaxel) in both taxane sensitive and taxane resistant/refractory human tumor cell lines. Briefly, compound 3102, paclitaxel and docetaxel were analyzed for their effects on proliferation on HCT116 and HT-29 colon carcinomas, the DLD-1 resistant colon carcinoma, PANC-1 pancreatic adenocarcinoma, PC-3 and LNCaP prostate carcinomas, IA9 ovarian carcinoma, and the paclitaxel resistant 1A9-PTX10 and 1A9-PTX22 ovarian carcinomas. All cell lines were maintained in RPMI-1640 tissue culture medium (TCM) (supplemented with antibiotics and 10% fetal bovine serum) and cultured at 37° C. in humidified air containing 5% CO₂. To assess the antiproliferative effects of test compounds, tumor cell cultures were first established at 1×10⁴ cells/ml in tissue culture medium and incubated for 24 hr at 37° C. in 10% CO₂ in air in order to allow cells to attach. A volume of 200 μl of medium was removed from each test well and 200 μl of medium containing dilutions (0.1, 1.0, 10.0, 100 nM) of the test agent (dissolved in TCM and 0.1% DMSO) was added to each well containing tumor cells and the resulting test plate incubated for 72 hr. Following incubation, IC₅₀ values were determined by adding 75 μL of warm growth media containing 5 mg/mL MTT (3-[4,5-dimethylthiazol-2-yl]-2,5-diphenyltetrazolium bromide) to each well and the cultures returned to the incubator, and left undisturbed for 1 hr. Plates were processed and the absorbance of the resulting solutions was measured by a plate reader at 570 nm. The absorbance of test wells was divided by the absorbance of drug-free wells, and the concentration of agent that resulted in 50% of the absorbance of untreated cultures (IC₅₀) was determined by analyses of best fit curve of the data. The results of this study (summarized in Table 2 below) show that compound 3102 retains good potency in various human tumor cell lines including the DLD-1 colon carcinoma which overexpresses p-glycoprotein and which is resistant to both paclitaxel and docetaxel. Compound 3102 is at least 5-fold more potent compared to both paclitaxel and docetaxel in killing DLD-1 tumor cells in vitro. In the ovarian cancer cell lines, 1A9-PTX10 and 1A9-PTX22 which have been made paclitaxel resistant due to a specific tubulin mutation (1A9-PTX10 Phe->Ala at β270, 1A9-PTX22 Ala->Thr at β364), the antitumor activity of compound 3102 was at least 4 to 8 fold more potent compared to that of paclitaxel. In general, IC₅₀ values of compound 3102 for all cell lines tested were equivalent or slightly superior to those obtained with docetaxel. These results indicate that the in vitro antitumor activity of compound 3102 is superior to that of paclitaxel and that the compound is capable of overcoming paclitaxel resistance mediated by two diverse types of mechanisms in tumor cells, those being overexpression of p-glycoprotein and specific tubulin mutations. The in vitro antitumor activity compound 3102 is at the very least equivalent, or in many cases, superior to that of docetaxel in the cell lines tested.

TABLE 2
In Vitro Antitumor Activity of Compound 3102 Compared
to Paclitaxel and Docetaxel

| | Tumor | Resistance | | | |
|---|---|---|---|---|---|
| Cell Line | Origin | Mech. | 3102 | Paclitaxel | Docetaxel |
| HCT-116 | Colon | — | 0.9 | 2.4 | 0.9 |
| HT-29 | Colon | — | 1.2 | 2.6 | 1.2 |
| DLD-1 | Colon | p-glycoprotein | 1.9 | >10 | 9.2 |
| PC-3 | Prostate | — | 1.8 | 4.1 | 2.8 |
| LnCaP | Prostate | — | 2.6 | 3.1 | 1.9 |
| PANC-1 | Pancreatic | — | 1.8 | 4.2 | 2.0 |
| 1A9 | Ovarian | — | 1.6 | 2.5 | 1.8 |
| 1A9- | Ovarian | Tubulin mutation | 5.2 | >40 | 7.0 |
| PTX10 | | | | | |
| 1A9- | Ovarian | Tubulin mutation | 8.5 | >30 | 8.5 |
| PTX22 | | | | | |

### EXAMPLE 5

### In Vivo Activity of Compound 3102 in Nude Mice Bearing Human Tumor Xenografts

Compound 3102 was investigated for its in vivo antitumor activity in a number of experimental tumor models. The models consisted of human tumors implanted into nude mice (human tumor xenografts). The models represented human cancers such as colon (HT-29, DLD-1 and SW480), pancreatic (Panc-1) melanoma (A375), renal (786-0) and mesothelioma (MSTO-211H). Studies were carried out at Piedmont Research Center, Morrisville, N.C. (HT-29, Panc-1, DLD-1, A375 and 786-0) and at Taxolog, Inc., Tallahassee, Fla. (MSTO-211H). Initial studies concentrated on the HT-29 colon and Panc-1 pancreatic tumor models. In these studies, effective routes of administration (IV and oral) and dosing schedules were determined for compound 3102. In the later of these studies, comparisons were made with the antitumor activities of paclitaxel and docetaxel at their optimum dose and schedule. Studies were expanded to determine the efficacy of compound 3102 in additional models of colon (DLD-1, SW480) pancreatic (Panc-1), melanoma (A375), renal (786-0) and mesothelioma (MSTO-211H) cancers. The studies described show that compound 3102 is effective at both IV and oral dosing in dramatically slowing the growth of human tumor xenografts in nude mice.

### EXAMPLE 6

### In Vivo Activity of Compound 3102 in Nude Mice Bearing HT-29 Human Tumor Xenografts

The protocol for HT-29 human tumor xenograft studies is described as follows:

Mice

Female athymic nude mice (Harlan) were 13-14 weeks old on Day 1 of the study. The animals were fed ad libitum water (reverse osmosis, 1 ppm CI) and NIH 31 Modified and Irradiated Lab Diets consisting of 18.0% crude protein, 5.0% crude fat, and 5.0% crude fiber. The mice were housed on ALPHA-dri® bed-o-cobs® Laboratory Animal Bedding in static microisolators on a 12-hour light cycle at 21-22° C. (70-72° F.) and 40-60% humidity.

Tumor Implantation

The HT29 colon tumor line used for this study was maintained in athymic nude mice. A tumor fragment (1 mm³) was implanted s.c. into the right flank of each test mouse. Tumors were monitored twice weekly and then daily as their volumes approached 200-400 mm³. On Day 1 of the study, the animals were sorted into treatment groups with tumor sizes of 108.0-486.0 mm³ and group mean tumor sizes of 224.9-230.0 mm³. Tumor size, in mm³, was calculated from:

$$Tumor Volume = \frac{{w}^{2} \times l}{2}$$

where w=width and l=length in mm of the tumor. Tumor weight was estimated with the assumption that 1 mg is equivalent to 1 mm³ Of tumor volume.

Drugs

Compound 3102 (Lot # HN-4-95-4) and TL-2 (Taxotere®) (Lot # HN-4-8-2A) were provided by Taxolog. Compound 3102 was dissolved in 50% ethanol and 50% Cremophor® EL to prepare 10× stock solutions. These stock solutions were diluted with saline immediately prior to dosing to yield dosing solutions in a vehicle consisting of 5% ethanol, 5% Cremophor® EL, and 90% saline (5% E 5% C in saline) for oral administration. For intraveneous administration, compound 3102 was dissolved in 100% ethanol to prepare 20× stock solutions. These solutions were diluted with 20% Liposyn®II on each day of dosing to yield dosing solutions in a vehicle consisting of 5% ethanol and 95% Liposyn® II (5% E95% L-II). Paclitaxel (Mayne Group Ltd., formerly NaPro Biotherapeutics, Inc.) was dissolved in 50% ethanol and 50% Cremophor® EL to prepare a 10× stock solution. On each day of dosing, an aliquot of the stock solution was diluted with 5% dextrose in water (D5W, pH ˜4.8) to yield a dosing solution containing 5% ethanol, 5% Cremophor® EL, and 90% D5W. Taxotere® was dissolved in 50% ethanol and 50% Tween® 80 to prepare a 6.67× stock solution. The Taxotere® stock solution was diluted with D5W immediately prior to dosing to yield a dosing solution in a vehicle consisting of 7.5% ethanol, 7.5% Tween® 80, and 85% D5W (7.5% E 7.5% T in D5W).

Treatment

Mice were sorted into appropriate groups with six mice per group, and treated in accordance with the protocol for each study. Some studies included Taxotere® (TL-2), and paclitaxel groups as positive drug controls. Taxotere® and paclitaxel were always administered at their optimum dose (30 mg/kg for both Taxotere® and paclitaxel), route (intravenously, IV) and schedule (weekly for three cycles, Q7Dx3 for Taxotere® and every other day for five cycles, QODx5 for paclitaxel). Administration of compound 3102 was either IV or oral (po). Control group mice received saline vehicle. Treatment schedules tested for compound 3102 were once daily (QDx1), every four days times four cycles (Q4Dx4), or every other day times five cycles (QODx5).

Endpoint

Each animal was euthanized when its neoplasm reached the predetermined endpoint size (1,000 mm³). The time to endpoint (TTE) for each mouse was calculated by the following equation:

$$\frac{TTE = {log}_{10} ( endpoint volume ) - b}{m}$$

where TTE is expressed in days, endpoint volume is in mm³, b is the intercept, and m is the slope of the line obtained by linear regression of a log-transformed tumor growth data set. The data set is comprised of the first observation that exceeded the study endpoint volume and the three consecutive observations that immediately preceded the attainment of the endpoint volume. Animals that do not reach the endpoint are assigned a TTE value equal to the last day of the study. Animals classified as treatment-related (TR) deaths or nontreatment-related metastasis (NTRm) deaths are assigned a TTE value equal to the day of death. Animals classified as non-treatment-related (NTR) deaths are excluded from TTE calculations.

Treatment efficacy was determined from tumor growth delay (TGD), which is defined as the increase in the median TTE for a treatment group compared to the control group:
TGD=T−C,
expressed in days, or as a percentage of the median TTE of the control group:

$$\% TGD = \frac{T - C}{C} \times 100$$

where:

T=median TTE for a treatment group,

C=median TTE for control Group 1.

Treatment may cause partial regression (PR) or complete regression (CR) of the tumor in an animal. In a PR response, the tumor volume is 50% or less of its Day 1 volume for three consecutive measurements during the course of the study, and equal to or greater than 13.5 mm³ for one or more of these three measurements. In a CR response, the tumor volume is less than 13.5 mm³ for three consecutive measurements during the course of the study. An animal with a CR response at the termination of a study is additionally classified as a long-term tumorfree survivor (LTTFS).

Mean Days of Survival

The mean days of survival (MDS) values were calculated for all groups. MDS values were the mean number of days required for the tumor to reach a specified weight (either 1.2 g or 2.0 g), depending on the study.

Statistical and Graphical Analyses

The logrank test was employed to analyze the significance of the difference between the TTE values of a drug-treated group and the vehicle-treated control group. The logrank test analyzes the data for all animals except the NTR deaths. The two-tailed statistical analyses were conducted at P=0.05, using Prism 3.03 (GraphPad) for Windows.

The tumor growth curves show the group median tumor volume as a function of time. When an animal exits the study due to tumor size or TR death, the final tumor volume recorded for the animal is included with the data used to calculate the median volume at subsequent time points. If more than one death occurs in a treatment group, the tumor growth curve for that group is truncated on the day of the last measurement that preceded the second death.

### EXAMPLE 7

### Study HT-29 e51 and e52: Initial Dosing and Scheduling Studies

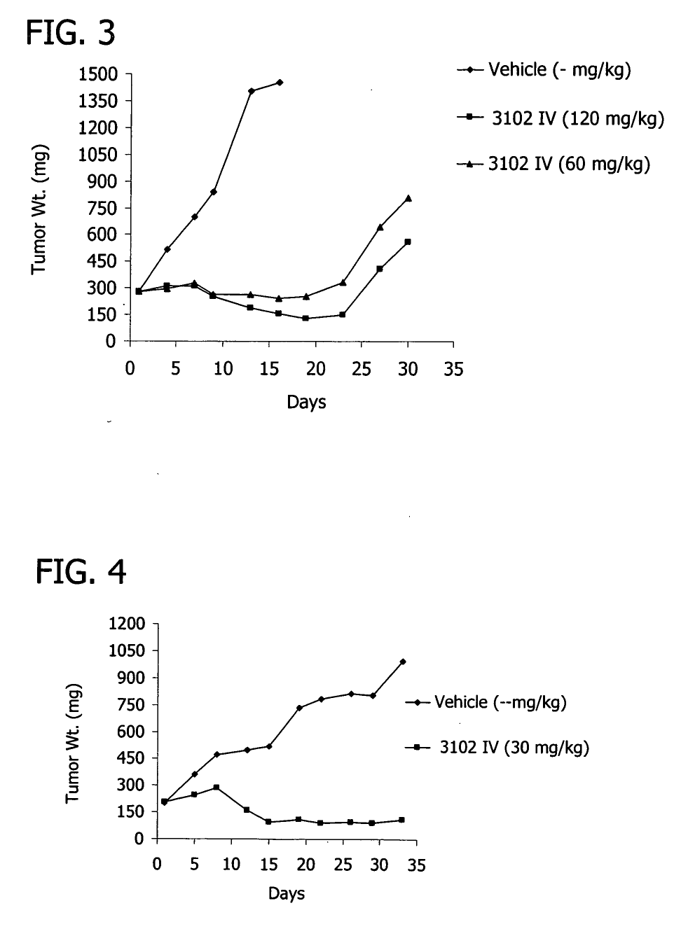

Studies were initiated to initially determine a route and schedule for administration of compound 3102 to HT-29 bearing mice. Compound 3102 was administered at 120 and 60 mg/kg on a QDx1 schedule (e52) and 30 mg/kg on a Q4Dx4 schedule (e51). The results of these studies are depicted in FIG. 3 and FIG. 4 and Tables 3 and 4.

FIG. 3 shows that compound 3102 administered intravenously at 120 and 60 mg/kg on a schedule of QDx1 is effective in controlling the growth of HT-29 tumor xenografts with a MDS of 38.5 and 32.4 for 120 and 60 mg/kg, respectively, compared to an MDS of only 12.1 days for vehicle treated mice. Maximum body weight loss in compound 3102 treated mice was minimal (−5.5% and −8.9% for 120 and 60 mg/kg treated mice, respectively) and occurred on Day 7 for both treatment groups.

TABLE 3
Treatment Response Summary for the HT29-e52 Study

| | | | | | | | Max. % | | | | | |
|---|---|---|---|---|---|---|---|---|---|---|---|---|
| | | | | | | MDS to | BW | | | | | |
| | | Regimen 1 | | | | 1.2 g ± SEM | Loss; | # Death^{a} | | | | |
| Grp | n | Agent | mg/kg | Route | Schedule | (n) | Day | TR | NTR | #CR | #PR | #SD/PD |
| 1 | 6 | Vehicle | — | IV | QD × 1 | 12.1 ± 0.8 (6) | — | 0 | 0 | 0 | 0 | 0 |
| 11 | 6 | 3102 | 120 | IV | QD × 1 | 38.5 ± 1.9 (6) | −5.5%; | 0 | 0 | 0 | 0 | 0 |
| | | | | | | | Day 7 | | | | | |
| 12 | 6 | 3102 | 60 | IV | QD × 1 | 32.4 ± 2.7 (4) | −8.9%; | 0 | 0 | 0 | 0 | 2 |
| | | | | | | | Day 7 | | | | | |

^{a}# Death: TR (Treatment Related); NTR (Non-Treatment Related)

TABLE 4
Treatment Response Summary for the HT29-e51 Study

| | | | | | | | Max. % | | |
|---|---|---|---|---|---|---|---|---|---|
| | | | | | | MDS to | BW | | |
| | | Regimen 1 | | | | 1.0 g ± SEM | Loss; | # Death^{a} | |
| Grp | n | Agent | mg/kg | Route | Schedule | (n) | Day | TR | NTR |
| 1 | 6 | 5% EC | — | IV | Q4D × 4 | 26.0 ± 5.5 (10) | — | 0 | 0 |
| | | in Saline | | | | | | | |
| 14 | 6 | 3102 | 30 | IV | Q4D × 4 | ± | −16.2%; | 1 | 0 |
| | | | | | | | Day 19 | | |

^{a}# Death: TR (Treatment Related); NTR (Non-Treatment Related)

At a dose of 30.0 mg, using a multi-dose schedule of Q4Dx4, compound 3102 was effective in controlling the growth of HT-29 xenografts for 33 days (FIG. 4). While vehicle treated animals 310 mice initially reached 300 mg on day 7, then fell to less than 150 mg for the remainder of the study. Maximum body weight loss was moderate (−16.2%) and occurred on Day 19. There was one treatment related death associated with this regimen (see Table 4).

These initial results indicated that compound 3102 was effective in slowing the growth of HT-29 human colon tumors as xenografts in nude mice. Compound 3102 could be effectively administered i.v. as both a single or multiple dose regimen, with little to moderate weight loss.

### EXAMPLE 8

### Study Ht-29 e60 and e76: Initial Single Vs. Multiple Dosing (Oral)

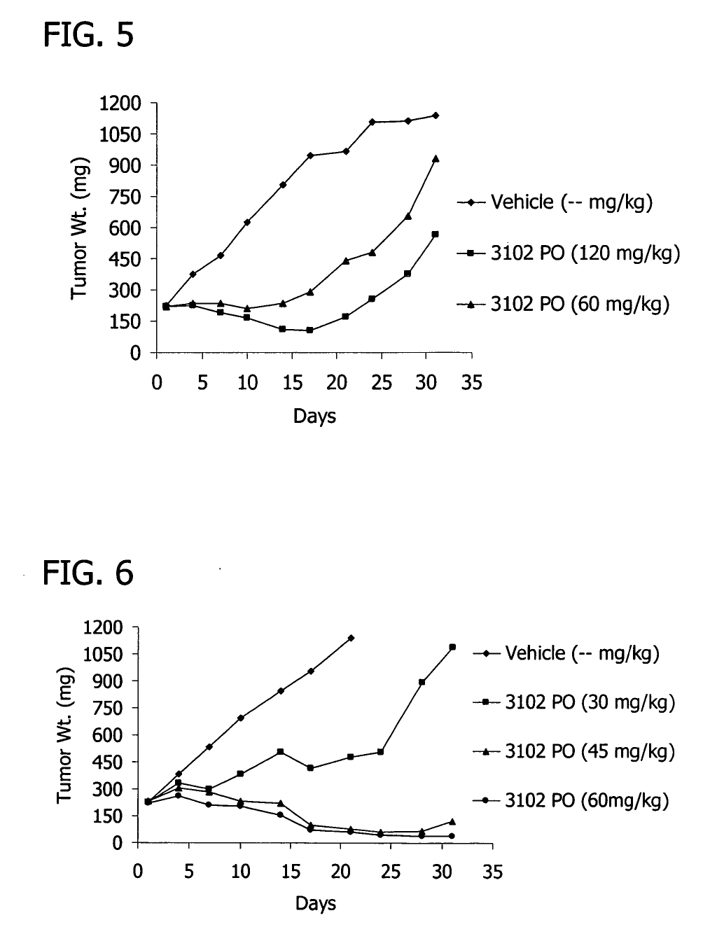

Compound 3102 was initially evaluated in the HT-29 xenograft model for both single oral dose (QDx1) at 60 and 120 mg/kg and multiple oral dose (Q4Dx4) at 30, 45, and 60 mg/kg. The results are presented in FIG. 5 and FIG. 6 and Tables 5 and 6. The results of these studies show that compound 3102, when given orally at a single dose, was effective in controlling the growth of HT-29 tumors, at a dosage of 120 mg/kg and 60 mg/kg (FIG. 5) compared to vehicle control. The MDS values for the 120 and 60 mg/kg dose were 35.3 and 31.8 days, respectively, compared to only 16.5 days for vehicle treated mice. Maximum body weight loss was observed on day 7 and for only the 120 mg/kg dose group and was minimal (5.5%).

TABLE 5
Treatment Response Summary for the HT29-e60 Study

| | | | | | | | Max. % | | | | | |
|---|---|---|---|---|---|---|---|---|---|---|---|---|
| | | | | | | MDS to | BW | | | | | |
| | | Regimen 1 | | | | 1.0 g ± SEM | Loss; | # Death^{a} | | | | |
| Grp | n | Agent | mg/kg | Route | Schedule | (n) | Day | TR | NTR | #CR | #PR | #SD/PD |
| 1 | 6 | 5% EC | — | PO | QD × 1 | 16.5 ± 2.5 (5) | — | 0 | 0 | 0 | 0 | 1 |
| | | in Saline | | | | | | | | | | |
| 2 | 6 | 3102 | 120 | PO | QD × 1 | 35.3 ± 2.9 (3) | −5.5%; | 0 | 0 | 0 | 0 | 3 |
| | | | | | | | Day 7 | | | | | |
| 3 | 6 | 3102 | 60 | PO | QD × 1 | 31.8 ± 1.9 (5) | — | 0 | 0 | 0 | 0 | 1 |

^{a}# Death: TR (Treatment Related); NTR (Non-Treatment Related)

TABLE 6
Treatment Response Summary for the HT29-e76 Study

| | | | | | | | Max. % | | | | | |
|---|---|---|---|---|---|---|---|---|---|---|---|---|
| | | | | | | MDS to | BW | | | | | |
| | | Regimen 1 | | | | 1.0 g ± SEM | Loss; | # Death^{a} | | | | |
| Grp | n | Agent | mg/kg | Route | Schedule | (n) | Day | TR | NTR | #CR | #PR | #SD/PD |
| 1 | 6 | 5% EC | — | PO | QD × 1 | 16.5 ± 1.5 (6) | — | 0 | 0 | 0 | 0 | 0 |
| | | in Saline | | | | | | | | | | |
| 9 | 6 | 3102 | 30 | PO | Q4D × 4 | 27.9 ± 1.0 (5) | −7.4%; | 0 | 0 | 0 | 1 | 0 |
| | | | | | | | Day 17 | | | | | |
| 10 | 6 | 3102 | 45 | PO | Q4D × 4 | ±(0) | −12.8%; | 0 | 0 | 1 | 3 | 2 |
| | | | | | | | Day 14 | | | | | |
| 11 | 6 | 3102 | 60 | PO | Q4D × 4 | 14.0 ± (1) | −17.8%; | 0 | 0 | 0 | 5 | 0 |
| | | | | | | | Day 17 | | | | | |

^{a}# Death: TR (Treatment Related); NTR (Non-Treatment Related)

The results from the oral, multi-dose study were even more encouraging. The results from this study show that a Q4Dx4 schedule of compound 3102 was highly effective in preventing growth of HT-29 tumors. Mice treated with a dose as low as 30 mg/kg had an MDS of 27.9 days, compared to 16.5 days for vehicle treated controls, with moderate maximum body weight loss (−7.4%). Mice treated with 45 mg/kg never grew out tumors, and this dose was associated with 1 complete response and three partial responses. Mice treated at the high dose (60 mg/kg) were associated with 5 partial responses, but no MDS values could be calculated as one mouse from the group grew tumor, with an MDS value of 14.0 days. The results of these studies indicate that compound 3102 can be administered orally, at both a single and multi-dose schedule, which effectively controls HT-29 tumor growth in mice.

### EXAMPLE 9

### Study HT-29 e103: Follow Up Studies for Single Dosing (Oral)

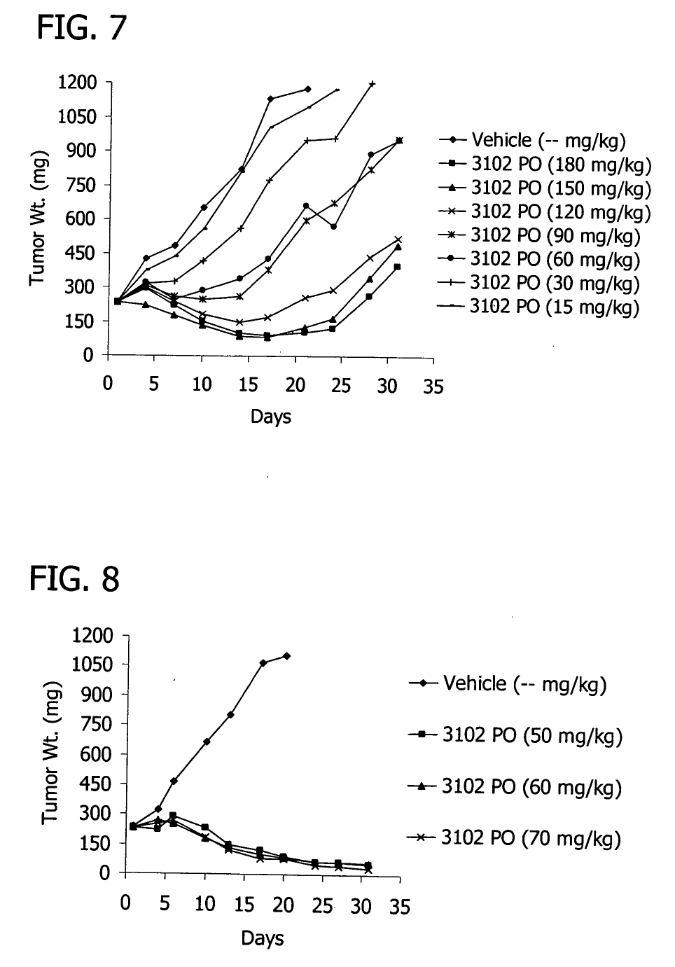

A follow-up study was initiated to determine the range of effective dosing for single, oral dosing in the HT-29 xenografts. The following doses were evaluated, 180, 150, 120, 90, 60, 30 and 15 mg/kg. The results of these studies are presented in FIG. 7 and Table 7. Treatment at the three higher doses resulted in a substantial delay in the growth of HT-29 tumors (MDS of 41.8, 42.1 and 40.5 for 180, 150 and 120 mg/kg, respectively) as compared to vehicle treated controls (MDS of 14.8 days). Partial responses were observed at 120 and 60 mg/kg. Body weight losses were negligible at all doses tested. The results of this study indicate that high doses of compound 3102 are tolerated well in mice and are associated with excellent anti-tumor efficacy in HT-29 implanted tumors.

TABLE 7
Treatment Response Summary for the HT29-e 103 Study

| | | | | | | | Max. % | | | | | |
|---|---|---|---|---|---|---|---|---|---|---|---|---|
| | | | | | | MDS to | BW | | | | | |
| | | Regimen 1 | | | | 1.0 g ± SEM | Loss; | # Death^{a} | | | | |
| Grp | n | Agent | mg/kg | Route | Schedule | (n) | Day | TR | NTR | #CR | #PR | #SD/PD |
| 1 | 6 | 5% EC | — | PO | QD × 1 | 14.8 ± 1.2 (6) | — | 0 | 0 | 0 | 0 | 0 |
| | | in Saline | | | | | | | | | | |
| 9 | 6 | 3102 | 180 | PO | QD × 1 | 41.8 ± 1.8 (6) | — | 0 | 0 | 0 | 0 | 0 |
| 10 | 6 | 3102 | 150 | PO | QD × 1 | 42.1 ± 2.6 (6) | −0.9%; | 0 | 0 | 0 | 0 | 0 |
| | | | | | | | Day 7 | | | | | |
| 11 | 6 | 3102 | 120 | PO | QD × 1 | 40.5 ± 2.8 (4) | — | 0 | 0 | 0 | 1 | 1 |
| 12 | 6 | 3102 | 90 | PO | QD × 1 | 31.4 ± 3.0 (6) | — | 0 | 0 | 0 | 0 | 0 |
| 13 | 6 | 3102 | 60 | PO | QD × 1 | 27.6 ± 3.2 (5) | — | 0 | 0 | 0 | 1 | 0 |
| 14 | 6 | 3102 | 30 | PO | QD × 1 | 21.5 ± 2.0 (6) | — | 0 | 0 | 0 | 0 | 0 |
| 15 | 6 | 3102 | 15 | PO | QD × 1 | 17.1 ± 1.8 (6) | — | 0 | 0 | 0 | 0 | 0 |

^{a}# Death: TR (Treatment Related); NTR (Non-Treatment Related)

### EXAMPLE 10

### Study HT-29 e79 and e80: Follow Up Studies for Multi-Dosing, Q4Dx4 and Q7Dx3 (Oral)

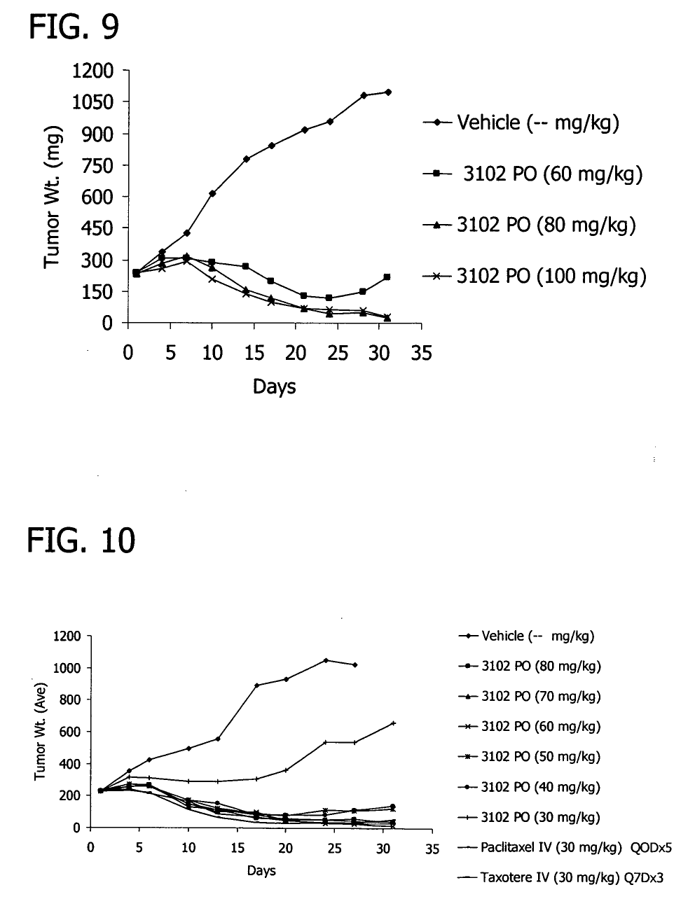

Studies were initiated to further investigate the efficacy of oral multi-dosing of compound 3102 in HT-29 tumor xenografts (studies e79 and e80). Two dosing schedules were evaluated, Q4Dx4 and Q7Dx3. The results are presented in FIG. 8 and FIG. 9 and Tables 8 and 9. Compound 3102, multi-d dosed orally at 70, 60 and 50 mg/kg was effective in slowing the growth and reducing the tumor volume of HT-29 tumors implanted in nude mice (FIG. 8). In the two higher dosage groups (70 and 60 mg/kg), compound 3102 treatment resulted in partial regressions of 6/6 in each group, while the lowest dosage tested (50 mg/kg) resulted in 4/6 partial regressions. All doses were associated with low to moderate body weight loss (Table 8). In the Q7Dx3 multi-dose group, compound 3102 treatment at the two highest doses resulted in 5/6 partial regressions at 100 mg/kg and 3/6 partial and 2/6 complete regressions in the 80 mg/kg group. The highest body weight loss occurred in the high dose group, but this did not exceed 10% (Table 9). The results of these studies indicate that multi-dosing with either Q4Dx4 or Q7Dx3 of orally administered compound is highly efficacious and well tolerated in mice.

TABLE 8
Treatment Response Summary for the HT29-e79 Study

| | | | | | | | Max. % | | | | | |
|---|---|---|---|---|---|---|---|---|---|---|---|---|
| | | | | | | MDS to | BW | | | | | |
| | | Regimen 1 | | | | 1.0 g ± SEM | Loss; | # Death^{a} | | | | |
| Grp | n | Agent | mg/kg | Route | Schedule | (n) | Day | TR | NTR | #CR | #PR | #SD/PD |
| 1 | 6 | 5% EC | — | PO | QD × 1 | 15.5 ± 0.8 (6) | — | 0 | 0 | 0 | 0 | 0 |
| | | in Saline | | | | | | | | | | |
| 5 | 6 | 3102 | 50 | PO | Q4D × 4 | ±(0) | −0.9%; | 0 | 0 | 0 | 4 | 2 |
| | | | | | | | Day 10 | | | | | |
| 6 | 6 | 3102 | 60 | PO | Q4D × 4 | ±(0) | −6.5%; | 0 | 0 | 0 | 6 | 0 |
| | | | | | | | Day 17 | | | | | |
| 7 | 6 | 3102 | 70 | PO | Q4D × 4 | ±(0) | −6.1%; | 0 | 0 | 0 | 6 | 0 |
| | | | | | | | Day 10 | | | | | |

^{a}# Death: TR (Treatment Related); NTR (Non-Treatment Related)

TABLE 9
Treatment Response Summary for the HT29-e80 Study

| | | | | | | | Max. % | | | | | |
|---|---|---|---|---|---|---|---|---|---|---|---|---|
| | | | | | | MDS to | BW | | | | | |
| | | Regimen 1 | | | | 1.0 g ± SEM | Loss; | # Death^{a} | | | | |
| Grp | n | Agent | mg/kg | Route | Schedule | (n) | Day | TR | NTR | #CR | #PR | #SD/PD |
| 1 | 6 | 5% EC | — | | QD × 1 | 19.5 ± 3.6 (6) | — | 0 | 0 | 0 | 0 | 0 |
| | | in Saline | | | | | | | | | | |
| 5 | 6 | 3102 | 60 | PO | Q7D × 3 | 45.0 ± 6.8 (3) | −3.8%; | 0 | 0 | 0 | 0 | 3 |
| | | | | | | | Day 14 | | | | | |
| 6 | 6 | 3102 | 80 | PO | Q7D × 3 | ±(0) | −0.9%; | 0 | 0 | 2 | 3 | 1 |
| | | | | | | | Day 14 | | | | | |
| 7 | 6 | 3102 | 100 | PO | Q7D × 3 | ±(0) | −9.2%; | 0 | 0 | 0 | 5 | 1 |
| | | | | | | | Day 17 | | | | | |

^{a}# Death: TR (Treatment Related); NTR (Non-Treatment Related)

### EXAMPLE 11

### Study HT-29 e105: Multi-Dosing, Q4Dx4 and Q7Dx3 for Compound 3102 (Oral) and Comparison to Paclitaxel (IV) and Taxotere® (IV)

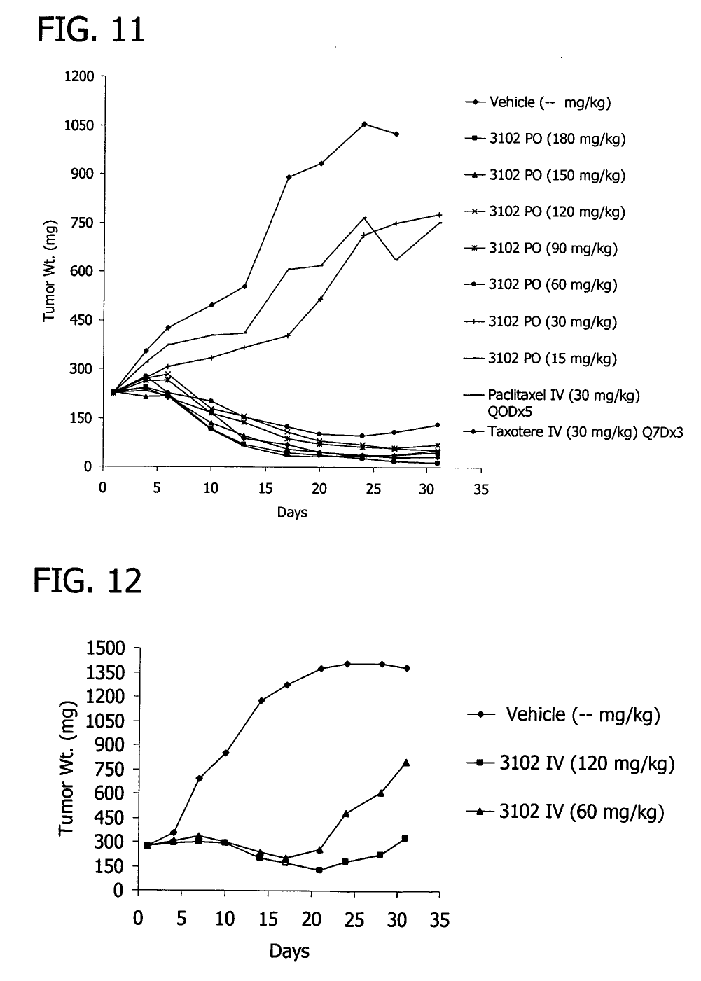

Multi-dosing studies with orally administered compound 3102 at two dosing schedules, Q4Dx4 and Q4Dx3 were undertaken to compare compound 3102's efficacy at various doses with that of paclitaxel and Taxotere® at their respective optimal dosing and schedules in the HT-29 tumor xenograft model (study e105). Results are presented in FIGS. 10 and 11 and Tables 10 and 11. Orally administered compound 3102 was effective at all doses tested in slowing the growth of HT-29 tumors, and reducing initial implant size at all doses except for the lowest dose (30 mg/kg) on a Q4Dx4 schedule. While both paclitaxel and Taxotere® were equally efficacious with orally administered compound 3102 at their optimal dose and schedules, body weight loss for Taxotere®) treated animals exceeded that observed for all doses of compound 3102 (Table 10).

On a Q7Dx3 schedule, all doses of orally administered compound 3102 resulted in dramatic slowing of the growth of HT-29 tumors and reducing tumor implant size (shrinking established tumors) except for the two lowest doses (30.0 mg/kg and 15.0 mg/kg). Both paclitaxel and Taxotere® were equally efficacious with orally administered compound 3102, however, as in the previous study, Taxotere® treated animals experienced severe weight loss at a level which was only exceeded by the highest dose of compound 3102 tested (180 mg/kg) (Table 11).

The results of these two studies show that orally administered compound 3102 is as efficacious as intravenously administered paclitaxel or Taxotere® (at their respective optimal dose and schedule) in treating HT-29 tumors in mice. In addition, compound 3102 is relatively non-toxic at the therapeutic doses given, as indicated by moderate body weight loss at all doses given except for the highest dose. This is in contrast to the body weight loss exhibited by Taxotere® treated mice in this model.

TABLE 10
Treatment Response Summary for the HT29-e105 Study

| | | | | | | | Max. % | | |
|---|---|---|---|---|---|---|---|---|---|
| | | | | | | MDS to | BW | | |
| | | Regimen 1 | | | | 1.0 g ± SEM | Loss; | # Death^{a} | |
| Grp | n | Agent | mg/kg | Route | Schedule | (n) | Day | TR | NTR |
| 1 | 6 | 5% EC | — | | | 19.0 ± 2.1 (10) | — | 0 | 0 |
| | | in Saline | | | | | | | |
| 9 | 6 | 3102 | 80 | PO | Q4D × 4 | ± | −15.4%; | 0 | 0 |
| | | | | | | | Day 17 | | |
| 10 | 6 | 3102 | 70 | PO | Q4D × 4 | ± | −13.2%; | 0 | 0 |
| | | | | | | | Day 17 | | |
| 11 | 6 | 3102 | 60 | PO | Q4D × 4 | ± | −13.5%; | 0 | 0 |
| | | | | | | | Day 17 | | |
| 12 | 6 | 3102 | 50 | PO | Q4D × 4 | 49.2± | −9.7%; | 0 | 0 |
| | | | | | | | Day 17 | | |
| 13 | 6 | 3102 | 40 | PO | Q4D × 4 | 44.9± | −9.1%; | 0 | 0 |
| | | | | | | | Day 17 | | |
| 14 | 6 | 3102 | 30 | PO | Q4D × 4 | 35.6 ± 5.8 | −2.7%; | 0 | 0 |
| | | | | | | | Day 17 | | |
| 21 | 6 | Paclitaxel | 30 | IV | QOD × 5 | ± | −7.3%; | 0 | 0 |
| | | | | | | | Day 13 | | |
| 22 | 6 | Taxotere | 30 | IV | Q7D × 3 | ± | −19.9%; | 0 | 0 |
| | | | | | | | Day 24 | | |

^{a}# Death: TR (Treatment Related); NTR (Non-Treatment Related)

TABLE 11
Treatment Response Summary for the HT29-e105 Study

| | | | | | | | Max. % | | |
|---|---|---|---|---|---|---|---|---|---|
| | | | | | | MDS to | BW | | |
| | | Regimen 1 | | | | 1.0 g ± SEM | Loss; | # Death^{a} | |
| Grp | n | Agent | mg/kg | Route | Schedule | (n) | Day | TR | NTR |
| 1 | 6 | 5% EC | — | PO | | 19.0 ± 2.1 (10) | — | 0 | 0 |
| | | in Saline | | | | | | | |
| 2 | 6 | 3102 | 180 | PO | Q7D × 3 | ± | −27.4%; | 2 | 0 |
| | | | | | | | Day 24 | | |
| 3 | 6 | 3102 | 150 | PO | Q7D × 3 | ± | −17.3%; | 0 | 0 |
| | | | | | | | Day 24 | | |
| 4 | 6 | 3102 | 120 | PO | Q7D × 3 | ± | −13.3%; | 0 | 0 |
| | | | | | | | Day 20 | | |
| 5 | 6 | 3102 | 90 | PO | Q7D × 3 | ± | −5.6%; | 0 | 0 |
| | | | | | | | Day 6 | | |
| 6 | 6 | 3102 | 60 | PO | Q7D × 3 | 48.8± | −5.9%; | 1 | 0 |
| | | | | | | | Day 6 | | |
| 7 | 6 | 3102 | 30 | PO | Q7D × 3 | 31.4 ± 5.8 | — | 0 | 0 |
| 8 | 6 | 3102 | 15 | PO | Q7D × 3 | 27.7 ± 4.6 | — | 0 | 0 |
| 21 | 6 | Paclitaxel | 30 | IV | QOD × 5 | ± | −7.3%; | 0 | 0 |
| | | | | | | | Day 13 | | |
| 22 | 6 | Taxotere | 30 | IV | Q7D × 3 | ± | −19.9%; | 0 | 0 |
| | | | | | | | Day 24 | | |

^{a}# Death: TR (Treatment Related); NTR (Non-Treatment Related)

### EXAMPLE 12

### Study Panc-1 e57, e59 and e92: Initial IV Dosing and Scheduling Studies

Similar anti-tumor efficacy studies as described for HT-29 were conducted with compound 3102 using Panc-1 human tumor xenografts in nude mice. The methods for conducting these experiments were identical to those for HT-29 except for the implant used.

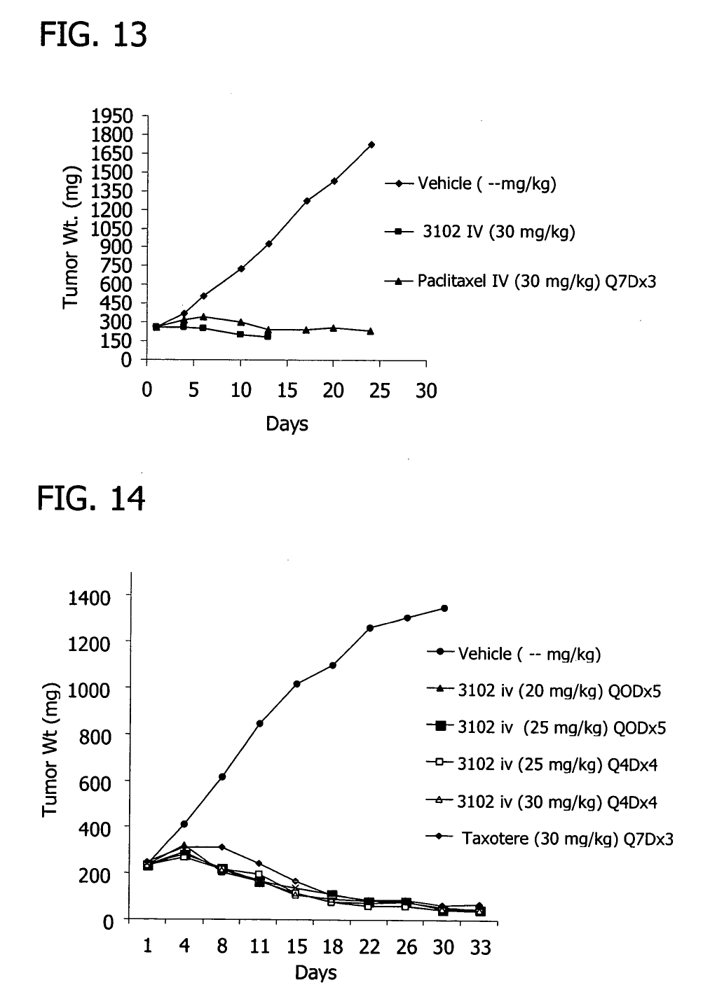

Studies were initiated to initially determine a route and schedule for administration of compound 3102 to Panc-1 bearing mice. Compound 3102 was administered intravenously at 120 and 60 mg/kg on a QDx1 schedule (e59) and 30 mg/kg on a multi-dose, QODx5 schedule (e57). Paclitaxel at its optimum dose (30 mg/kg) and schedule (QODx5) was also evaluated in the e57 study. The results of these studies are depicted in FIGS. 12 and 13 and Tables 12 and 13. Compound 3102 administered as a single, IV dose was effective in slowing the growth of Panc-1 human xenografts in nude mice compared to vehicle control (FIG. 12). MDS values for compound 3102 were 42.9 and 34.6 days for 120 mg/kg and 60 mg/kg, respectively, compared to 16.2 days for vehicle control. Only negligible body weight loss was observed at the highest dose of compound 3102.

For the multi-dose study, compound 3102 was administered, intravenously on a QODx5 schedule which is comparable to that of paclitaxel (FIG. 13). The results show that compound 3102 was effective early on in reducing tumor growth and initial implant size, however, the compound proved to be toxic for Panc-1 tumor implanted mice at the tested dose of 30 mg/kg as evidenced by severe body weight loss (Table 13). The results of these two studies demonstrate that compound 3102 can be administered intravenously at high dose (120 and 60 mg/kg) on a QDx1 schedule in mice bearing Panc-1 human tumor xenografts. Compound 3102 does not appear to be effective when administered intravenously on a dose and schedule comparable to that of paclitaxel (30 mg/kg, QODx5).

TABLE 12
Treatment Response Summary for the Panc-e59 Study

| | | | | | | | Max. % | | | | | |
|---|---|---|---|---|---|---|---|---|---|---|---|---|
| | | | | | | MDS to | BW | | | | | |
| | | Regimen 1 | | | | 1.2 g ± SEM | Loss; | # Death^{a} | | | | |
| Grp | n | Agent | mg/kg | Route | Schedule | (n) | Day | TR | NTR | #CR | #PR | #SD/PD |
| 1 | 6 | Vehicle | — | IV | QD × 1 | 16.2 ± 4.2 (6) | — | 0 | 0 | 0 | 0 | 0 |
| 11 | 6 | 3102 | 120 | IV | QD × 1 | 42.9 ± 2.8 (5) | −3.3%; | 1 | 0 | 0 | 0 | 0 |
| | | | | | | | Day 7 | | | | | |
| 12 | 6 | 3102 | 60 | IV | QD × 1 | 34.6 ± 1.4 (6) | — | 0 | 0 | 0 | 0 | 0 |

^{a}# Death: TR (Treatment Related); NTR (Non-Treatment Related)

TABLE 13
Treatment Response Summary for the Panc-e57 Study

| | | | | | | | Max. % | | |
|---|---|---|---|---|---|---|---|---|---|
| | | | | | | MDS to | BW | | |
| | | Regimen 1 | | | | 1.2 g ± SEM | Loss; | # Death^{a} | |
| Grp | n | Agent | mg/kg | Route | Schedule | (n) | Day | TR | NTR |
| 1 | 6 | Vehicle | — | | | 16.1 ± 1.7 (10) | — | 0 | 0 |
| 14 | 6 | 3102 | 30 | IV | QOD × 5 | ± | −22.1%; | 6 | 0 |
| | | | | | | | Day 10 | | |
| 19 | 6 | Paclitaxel | 30 | IV | Q7D × 3 | ± | −1.3%; | 0 | 0 |
| | | | | | | | Day 13 | | |

^{a}# Death: TR (Treatment Related); NTR (Non-Treatment Related)

### EXAMPLE 13

### Study Panc e92: Compound 3102 IV Multi-Dosing, Comparison of a Q4Dx4 to a QODx5 Schedule

An additional study was undertaken to compare the efficacy of intravenously administered compound 3102 given on a Q4Dx4, a QODx5 schedule and to Taxotere® given at its optimal dose and schedule. The results of this study are shown in FIG. 14 and Table 14. The QODx5 and Q4Dx4 schedules of intravenously administered compound 3102 resulted in complete control of tumor growth and shrinkage in tumor weight of the original tumor implant. While there were 2 partial responses and 1 complete response associated with the 20 mg/kg dose, the 25 mg/kg dose group experienced 4/6 treatment related deaths and moderate to severe body weight loss was observed with both doses. On the Q4Dx4 schedule, however, only a moderate body weight loss was associated with both treatment groups (25 and 30 mg/kg) and 5 complete responses and 1 complete response were observed for each group. Taxotere® treated animals experienced a similar reduction in tumor growth and tumor volume, with moderate body weight loss. These results clearly show that for intravenously dosed compound 3102, a schedule of Q4Dx4 and an appropriate dose level contributes to an impressive efficacy and low toxicity observed in the Panc-1 human tumor xenograft model.

TABLE 14
Treatment Response for the Panc-e92 Study

| | | | | | | | Max. % | | | | | |
|---|---|---|---|---|---|---|---|---|---|---|---|---|
| | | | | | | MDS to | BW | | | | | |
| | | Regimen 1 | | | | 1.2 g ± SEM | Loss; | # Death^{a} | | | | |
| Grp | n | Agent | mg/kg | Route | Schedule | (n) | Day | TR | NTR | #CR | #PR | #SD/PD |
| 1 | 6 | 5% | — | IV | QD × 1 | 17.0 ± 3.0 (6) | — | 0 | 0 | 0 | 0 | 0 |
| | | E95% LII | | | | | | | | | | |
| 11 | 6 | 3102 | 20 | IV | QOD × 5 | 59.6 ± 1.7 (3) | −13.7%; | 0 | 0 | 1 | 2 | 0 |
| | | | | | | | Day 11 | | | | | |
| 12 | 6 | 3102 | 25 | IV | QOD × 5 | ±(0) | −15.8%; | 4 | 0 | 0 | 2 | 0 |
| | | | | | | | Day 11 | | | | | |
| 13 | 6 | 3102 | 25 | IV | Q4D × 4 | ±(0) | −6.1%; | 0 | 0 | 1 | 5 | 0 |
| | | | | | | | Day 18 | | | | | |
| 14 | 6 | 3102 | 30 | IV | Q4D × 4 | ±(0) | −6.0%; | 0 | 0 | 1 | 5 | 0 |
| | | | | | | | Day 18 | | | | | |
| 22 | 6 | Taxotere | 30 | IV | Q7D × 3 | ±(0) | −9.6%; | 0 | 0 | 0 | 5 | 1 |
| | | | | | | | Day 22 | | | | | |

^{a}# Death: TR (Treatment Related); NTR (Non-Treatment Related)

### EXAMPLE 14

### Study Panc-1 e64 and e93: Single Oral Dosing

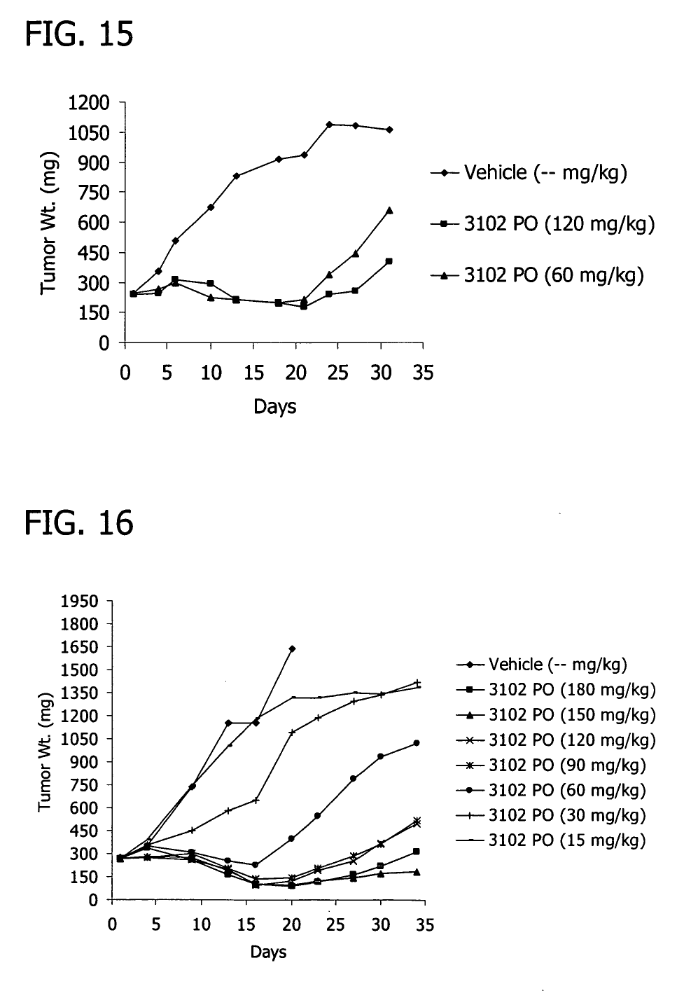

Compound 3102 was evaluated for efficacy in Panc-1 human tumor xenografts as a single dosing oral agent. Results of these studies are presented in FIGS. 15 and 16 and Tables 15 and 16. An initial study was carried out at two doses, 120 and 60 mg/kg to determine a range in which oral compound 3102 would be efficacious (study e64). FIG. 15 shows that both doses of compound 3102, when given as single dose, were able to dramatically reduce the tumor growth rate compared to vehicle control. MDS values were 44.6 days and 32.4 days for compound 3102 at 120 mg/kg and 60 mg/kg, respectively (Table 15). Only a negligible weight loss (−1.2%) was observed at the highest dose tested.

Based on the results of the e64 study, an additional study was designed to determine a maximum and minimum efficacious dose for orally administered compound 3102, single dose. The results of that study are presented in FIG. 16 and Table 16. Compound 3102 could be administered orally as a single dose up to 180 mg/kg without evidence of severe weight loss. Compound 3102 was clearly efficacious at all doses tested, even at the lower 30 and 15 mg/kg dose levels, with MDS values which exceeded that of the vehicle control. Partial regressions were observed at the three top doses 180, 150 and 120 mg/kg (1, 2 and 2, respectively). One treatment related death was observed at 60 mg/kg. These results indicate that compound 3102, when given as a single, oral dose, is highly efficacious in the treatment of Panc-1 tumors in mice.

TABLE 15
Treatment Response Summary for the Panc-e64 Study

| | | | | | | | Max. % | | | | | |
|---|---|---|---|---|---|---|---|---|---|---|---|---|
| | | | | | | MDS to | BW | | | | | |
| | | Regimen 1 | | | | 1.2 g ± SEM | Loss; | # Death^{a} | | | | |
| Grp | n | Agent | mg/kg | Route | Schedule | (n) | Day | TR | NTR | #CR | #PR | #SD/PD |
| 1 | 6 | 5% EC | — | PO | QD × 1 | 14.2 ± 3.5 (4) | — | 0 | 0 | 1 | 0 | 1 |
| | | in Saline | | | | | | | | | | |
| 2 | 6 | 3102 | 120 | PO | QD × 1 | 46.6 ± 5.5 (5) | −1.2%; | 0 | 0 | 0 | 1 | 0 |
| | | | | | | | Day 4 | | | | | |
| 3 | 6 | 3102 | 60 | PO | QD × 1 | 32.4 ± 2.7 (3) | — | 1 | 0 | 1 | 1 | 0 |

^{a}# Death: TR (Treatment Related); NTR (Non-Treatment Related)

TABLE 16
Treatment Response Summary for the Panc-e93 Study

| | | | | | | | Max. % | | | | | |
|---|---|---|---|---|---|---|---|---|---|---|---|---|
| | | | | | | MDS to | BW | | | | | |
| | | Regimen 1 | | | | 1.2 g ± SEM | Loss; | # Death^{a} | | | | |
| Grp | n | Agent | mg/kg | Route | Schedule | (n) | Day | TR | NTR | #CR | #PR | #SD/PD |
| 1 | 6 | 5% EC | — | PO | QD × 1 | 15.1 ± 1.2 (6) | — | 0 | 0 | 0 | 0 | 0 |
| | | in Saline | | | | | | | | | | |
| 9 | 6 | 3102 | 180 | PO | QD × 1 | 48.6 ± 3.1 (4) | −2.6%; | 0 | 0 | 0 | 1 | 1 |
| | | | | | | | Day 5 | | | | | |
| 10 | 6 | 3102 | 150 | PO | QD × 1 | ±(0) | −3.3%; | 0 | 0 | 0 | 2 | 4 |
| | | | | | | | Day 5 | | | | | |
| 11 | 6 | 3102 | 120 | PO | QD × 1 | 41.7 ± 1.7 (4) | −1.7%; | 0 | 0 | 0 | 2 | 0 |
| | | | | | | | Day 5 | | | | | |
| 12 | 6 | 3102 | 90 | PO | QD × 1 | 41.2 ± 1.9 (4) | −2.5%; | 0 | 0 | 0 | 0 | 2 |
| | | | | | | | Day 5 | | | | | |
| 13 | 6 | 3102 | 60 | PO | QD × 1 | 36.5 ± 5.0 (5) | −1.8%; | 1 | 0 | 0 | 0 | 0 |
| | | | | | | | Day 9 | | | | | |
| 14 | 6 | 3102 | 30 | PO | QD × 1 | 22.2 ± 2.5 (5) | — | 0 | 0 | 0 | 0 | 1 |
| 15 | 6 | 3102 | 15 | PO | QD × 1 | 17.4 ± 3.5 (6) | — | 0 | 0 | 0 | 0 | 0 |

^{a}# Death: TR (Treatment Related); NTR (Non-Treatment Related)

### EXAMPLE 15

### Study Panc-1 e79 and e87: Compound 3102, Multi-Dosing, Q4Dx4, Oral

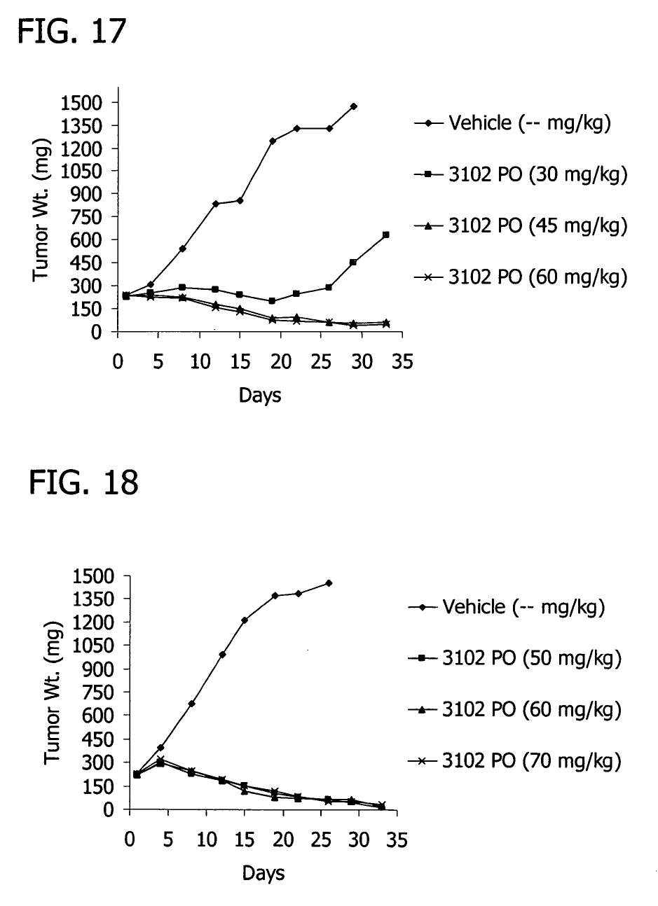

Multi-dosing studies with orally administered compound 3102 on a treatment schedule of Q4Dx4 were undertaken to compare compound 3102's efficacy in the Panc-1 tumor xenograft model (studies e79 and e87). These studies were aimed at determining starting dose levels and the data is presented in FIGS. 17 and 18 and Tables 17 and 18. The results for study e79 show that orally administered, compound 3102, on a schedule of Q4Dx4 was efficacious at all dose levels tested (FIG. 17), particularly at the two higher doses, 60 and 45 mg/kg, with 6/6 partial regressions noted for these doses (Table 17). The lower dose, 30 mg/kg, was associated with a slowing of Panc-1 tumor growth and 1 partial regression. A moderate body weight loss (−11.1%) was observed at the 60 mg/kg dose group. Study e87 further confirmed these results by demonstrating an even greater level of efficacy at a higher dose of 70 mg/kg (FIG. 18) which was associated mg/kg, were both associated with 6/6 partial regressions. Body weight loss was only moderate (−9.9%) which was associated with the 70 mg/kg dose group The data from studies e79 and e87 clearly demonstrate the effectiveness of orally administered compound 3102 given on a multi-dose schedule of Q4Dx4 with 1 complete regression and 5 partial regressions (Table 18). The remaining two doses tested, 50 and 60 mg/kg, were both associated with 6/6 partial regressions. Body weight loss was only moderate (−9.9%) which was associated with the 70 mg/kg dose group The data from studies e79 and e87 clearly demonstrate the effectiveness of orally administered compound 3102 given on a multi-dose schedule of Q4Dx4

TABLE 17
Treatment Response Summary for the Panc-e79 Study

| | | | | | | | Max. % | | | | | |
|---|---|---|---|---|---|---|---|---|---|---|---|---|
| | | | | | | MDS to | BW | | | | | |
| | | Regimen 1 | | | | 1.2 g ± SEM | Loss; | # Death^{a} | | | | |
| Grp | n | Agent | mg/kg | Route | Schedule | (n) | Day | TR | NTR | #CR | #PR | #SD/PD |
| 1 | 6 | 5% EC | — | PO | QD × 1 | 18.1 ± 2.2 (6) | — | 0 | 0 | 0 | 0 | 0 |
| | | in Saline | | | | | | | | | | |
| 9 | 6 | 3102 | 30 | PO | Q4D × 4 | 32.0 ± 2.7 (2) | −0.4%; | 0 | 0 | 0 | 1 | 3 |
| | | | | | | | Day 15 | | | | | |
| 10 | 6 | 3102 | 45 | PO | Q4D × 4 | ±(0) | −6.7%; | 0 | 0 | 0 | 6 | 0 |
| | | | | | | | Day 15 | | | | | |
| 11 | 6 | 3102 | 60 | PO | Q4D × 4 | ±(0) | −11.1%; | 0 | 0 | 0 | 6 | 0 |
| | | | | | | | Day 15 | | | | | |

^{a}# Death: TR (Treatment Related); NTR (Non-Treatment Related)

TABLE 18
Treatment Response Summary for the Panc-e87 Study

| | | | | | | | Max. % | | | | | |
|---|---|---|---|---|---|---|---|---|---|---|---|---|
| | | | | | | MDS to | BW | | | | | |
| | | Regimen 1 | | | | 1.2 g ± SEM | Loss; | # Death^{a} | | | | |
| Grp | n | Agent | mg/kg | Route | Schedule | (n) | Day | TR | NTR | #CR | #PR | #SD/PD |
| 1 | 6 | 5% EC | — | PO | QD × 1 | 15.5 ± 2.0 (6) | — | 0 | 0 | 0 | 0 | 0 |
| | | in Saline | | | | | | | | | | |
| 5 | 6 | 3102 | 50 | PO | Q4D × 4 | ±(0) | −1.4%; | 0 | 0 | 0 | 6 | 0 |
| | | | | | | | Day 12 | | | | | |
| 6 | 6 | 3102 | 60 | PO | Q4D × 4 | ±(0) | −2.7%; | 0 | 0 | 0 | 6 | 0 |
| | | | | | | | Day 12 | | | | | |
| 7 | 6 | 3102 | 70 | PO | Q4D × 4 | ±(0) | −9.9%; | 0 | 0 | 1 | 5 | 0 |
| | | | | | | | Day 12 | | | | | |

^{a}# Death: TR (Treatment Related); NTR (Non-Treatment Related)

### EXAMPLE 16

### Study Panc e95: Multi-Dosing, Q4Dx4 and Q7Dx3 for Compound 3102 (Oral) and Comparison to Paclitaxel (IV) and Taxotere® (IV)

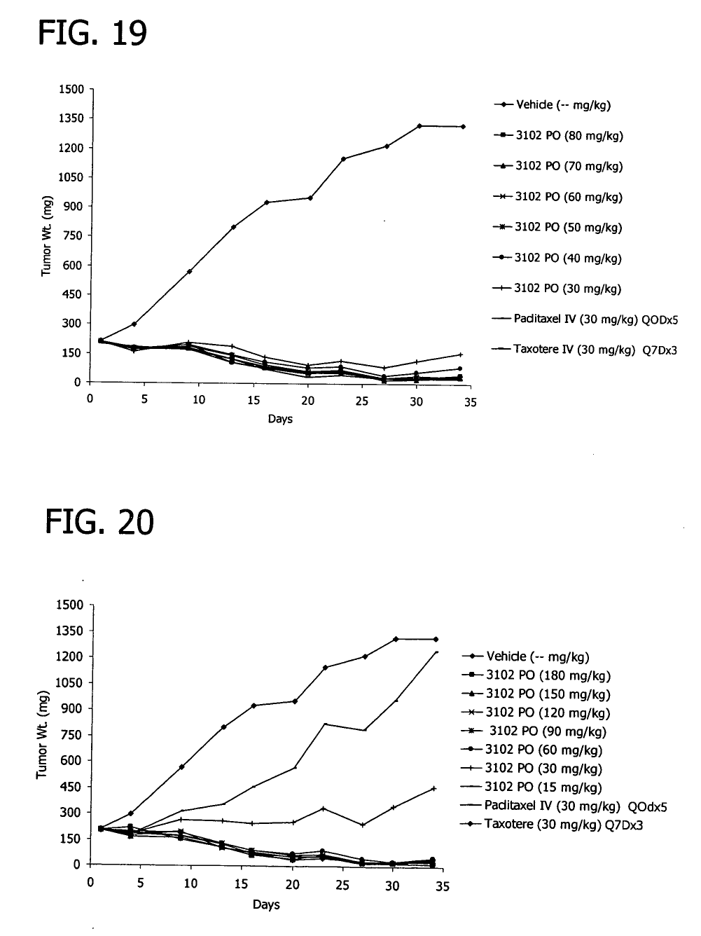

Multi-dosing studies with orally administered compound 3102 at two dosing schedules, Q4Dx4 and Q7Dx3 were undertaken to compare compound 3102's efficacy at various doses with that of paclitaxel and Taxotere® at their respective optimal dosing and schedules in the Panc-2 tumor xenograft model (study e95). Results are presented in FIGS. 19 and 20 and Tables 19 and 20. Orally administered compound 3102 was effective at all doses tested in slowing the growth of HT-29 tumors, and reducing initial implant size at all doses except for the lowest dose (30 mg/kg) on a Q4Dx4 schedule. While both paclitaxel and Taxotere® were equally efficacious with orally administered compound 3102 at their optimal dose and schedules, body weight loss for Taxotere® treated animals exceeded that observed for all doses of compound 3102 (Table 19).

On a Q7Dx3 schedule, all doses of orally administered compound 3102 resulted in dramatic slowing of the growth of HT-29 tumors and reducing tumor implant size (shrinking established tumors) except for the two lowest doses (30.0 mg/kg and 15.0 mg/kg). Both paclitaxel and Taxotere® were equally efficacious with orally administered compound 3102, however, as in the previous study, Taxotere® treated animals experienced severe weight loss at a level which was only exceeded by the highest dose of compound 3102 tested (180 mg/kg).

The results of these two studies show that orally administered compound 3102 is as efficacious as intravenously administered paclitaxel or Taxotere® (at their respective optimal dose and schedule) in treating HT-29 tumors in mice. In addition, compound 3102 is relatively non-toxic at the therapeutic doses given, as indicated by moderate body weight loss at all doses given except for the highest dose. This is in contrast to the body weight loss exhibited by Taxotere® treated mice in this model.

TABLE 19
Treatment Response Summary for the Panc-e95 Study

| | | | | | | | Max. % | | |
|---|---|---|---|---|---|---|---|---|---|
| | | | | | | MDS to | BW | | |
| | | Regimen 1 | | | | 1.2 g ± SEM | Loss; | # Death^{a} | |
| Grp | n | Agent | mg/kg | Route | Schedule | (n) | Day | TR | NTR |
| 1 | 6 | 5% EC | — | | | 24.4 ± 4.5 (10) | −1.2%; | 0 | 0 |
| | | in Saline | | | | | Day 2 | | |
| 9 | 6 | 3102 | 80 | PO | Q4D × 4 | ± | −9.7%; | 0 | 0 |
| | | | | | | | Day 13 | | |
| 10 | 6 | 3102 | 70 | PO | Q4D × 4 | ± | −11.7%; | 0 | 0 |
| | | | | | | | Day 16 | | |
| 11 | 6 | 3102 | 60 | PO | Q4D × 4 | ± | −8.2%; | 0 | 0 |
| | | | | | | | Day 13 | | |
| 12 | 6 | 3102 | 50 | PO | Q4D × 4 | ± | −2.5%; | 0 | 0 |
| | | | | | | | Day 16 | | |
| 13 | 6 | 3102 | 40 | PO | Q4D × 4 | 59.5± | −6.3%; | 0 | 0 |
| | | | | | | | Day 16 | | |
| 14 | 6 | 3102 | 30 | PO | Q4D × 4 | 49.8± | — | 0 | 0 |
| 21 | 6 | Paclitaxel | 30 | IV | QOD × 5 | ± | −2.6%; | 0 | 0 |
| | | | | | | | Day 13 | | |
| 22 | 6 | Docetaxel | 30 | IV | Q7D × 3 | ± | −21.6%; | 0 | 0 |
| | | | | | | | Day 27 | | |

^{a}# Death: TR (Treatment Related); NTR (Non-Treatment Related)

TABLE 20
Treatment Response Summary for the Panc-e95 Study

| | | | | | | | Max. % | | |
|---|---|---|---|---|---|---|---|---|---|
| | | | | | | MDS to | BW | | |
| | | Regimen 1 | | | | 1.2 g ± SEM | Loss; | # Death^{a} | |
| Grp | n | Agent | mg/kg | Route | Schedule | (n) | Day | TR | NTR |
| 1 | 6 | 5% EC | — | | | 24.4 ± 4.5 (10) | −1.2%; | 0 | 0 |
| | | in Saline | | | | | Day 2 | | |
| 2 | 6 | 3102 | 180 | PO | Q7D × 3 | ± | −26.9%; | 0 | 0 |
| | | | | | | | Day 27 | | |
| 3 | 6 | 3102 | 150 | PO | Q7D × 3 | ± | −25.8%; | 2 | 0 |
| | | | | | | | Day 27 | | |
| 4 | 6 | 3102 | 120 | PO | Q7D × 3 | ± | −12.0%; | 0 | 0 |
| | | | | | | | Day 23 | | |
| 5 | 6 | 3102 | 90 | PO | Q7D × 3 | ± | −14.8%; | 0 | 0 |
| | | | | | | | Day 20 | | |
| 6 | 6 | 3102 | 60 | PO | Q7D × 3 | ± | −4.5%; | 0 | 0 |
| | | | | | | | Day 13 | | |
| 7 | 6 | 3102 | 30 | PO | Q7D × 3 | 46.8 ± 3.6 | — | 0 | 0 |
| 8 | 6 | 3102 | 15 | PO | Q7D × 3 | 29.0 ± 2.2 | −1.3%; | 0 | 0 |
| | | | | | | | Day 13 | | |
| 21 | 6 | Paclitaxel | 30 | IV | QOD × 5 | ± | −2.6%; | 0 | 0 |
| | | | | | | | Day 13 | | |
| 22 | 6 | Docetaxel | 30 | IV | Q7D × 3 | ± | −21.6%; | 0 | 0 |
| | | | | | | | Day 27 | | |

^{a}# Death: TR (Treatment Related); NTR (Non-Treatment Related)

### EXAMPLE 17

### Study DLD e07: Compound 3102, Oral and Intravenous, Multi-Dose, Q4Dx4 with Paclitaxel and Taxotere® as Comparators

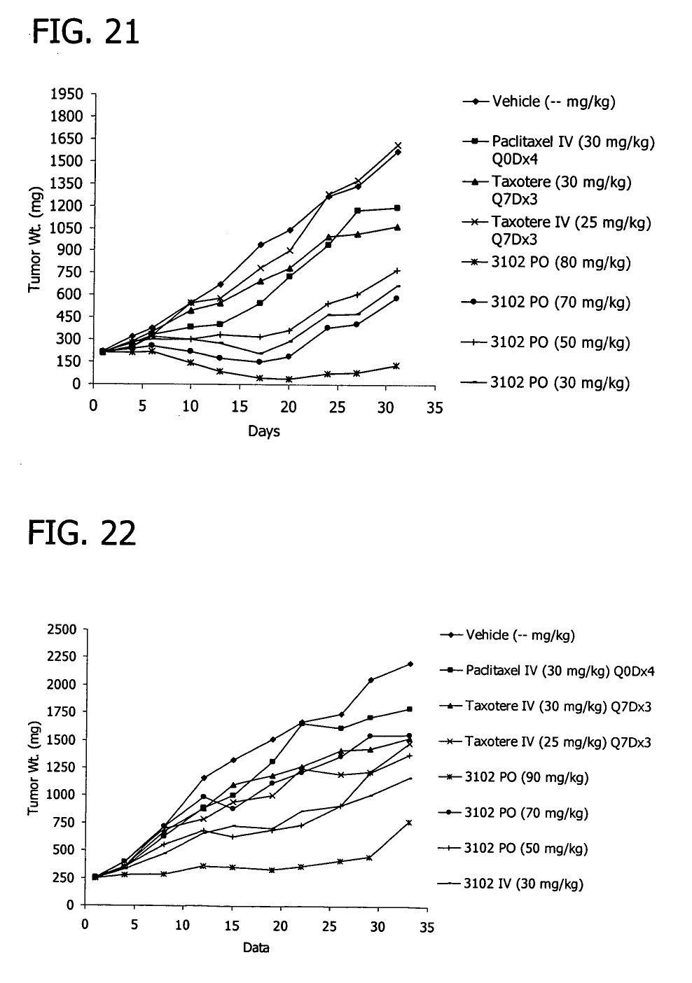

The multi-drug resistant, DLD-1 human colon carcinoma was used to evaluate the antitumor activities of orally and intravenously administered compound 3102 using a Q4Dx4 multi-dose schedule. Paclitaxel and Taxotere® were also evaluated in this model at their optimum dose, route (IV) and schedule. The results of this study are presented in FIG. 21 and Table 21. Oral compound 3102 was highly effective at all doses tested (80, 70 and 50 mg/kg) in reducing tumor growth in DLD-1 colon xenografts. The highest dose of compound 3102 tested, 80 mg/kg, was especially effective in reducing tumor weight to less than that of the initial implant. Compound 3102 at 35 mg/kg given intravenously was similarly effective in controlling tumor growth. Paclitaxel and Taxotere®, however, failed to demonstrate significant antitumor activity against DLD-1 tumors, with MDS values which were within the range of controls. The results of this study show that oral and iv administered compound 3102 is effective in the treatment of multi-drug resistant, DLD-1 colon tumors in mice.

TABLE 21
Treatment Response Summary for the DLD1-e07 Study

| | | | | | | | Max. % | | |
|---|---|---|---|---|---|---|---|---|---|
| | | | | | | MDS to | BW | | |
| | | Regimen 1 | | | | 2.0 g ± SEM | Loss; | # Death^{a} | |
| Grp | n | Agent | mg/kg | Route | Schedule | (n) | Day | TR | NTR |
| 1 | 6 | 5% EC | — | | | 37.6 ± 7.1 (10) | — | 0 | 0 |
| | | in Saline | | | | | | | |
| 3 | 6 | Paclitaxel | 30 | IV | QOD × 5 | 40.2 ± 4.7 | −0.9%; | 0 | 0 |
| | | | | | | | Day 6 | | |
| 4 | 6 | Taxotere | 30 | IV | Q7D × 3 | 37.6 ± 6.5 | −19.5%; | 0 | 0 |
| | | | | | | | Day 2 | | |
| 5 | 6 | Taxotere | 25 | IV | Q7D × 3 | 35.0 ± 2.3 | −20.8%; | 0 | |
| | | | | | | | Day 2 | | |
| 6 | 6 | 3102 | 80 | PO | Q4D × 4 | 53.8± | −16.4%; | 0 | 0 |
| | | | | | | | Day 1 | | |
| 7 | 6 | 3102 | 70 | PO | Q4D × 4 | 46.1 ± 3.5 | −13.5%; | 0 | 0 |
| | | | | | | | Day 1 | | |
| 8 | 6 | 3102 | 50 | PO | Q4D × 4 | 47.1 ± 3.9 | −17.4%; | 0 | 0 |
| | | | | | | | Day 2 | | |
| 12 | 6 | 3102 | 30 | IV | Q4D × 4 | 45.3 ± 4.6 | −12.0%; | 0 | 0 |
| | | | | | | | Day 1 | | |

### EXAMPLE 18

### Study SW480 e11: Compound 3102, Oral and Intravenous, Multi-Dose, Q4Dx4 with Paclitaxel and Taxotere® as Comparators

The SW480 human colon carcinoma was used to evaluate the antitumor activities of orally and intravenously administered compound 3102 using a Q4Dx4 multi-dose schedule. Paclitaxel and Taxotere® were also evaluated in this model at their optimum dose, route (IV) and schedule. The results of this study are presented in FIG. 22 and Table 22. Oral compound 3102 was effective at all doses tested (90, 70 and 50 mg/kg) in reducing tumor growth in SW480 colon xenografts. The highest dose of compound 3102 tested, 90 mg/kg, was especially effective in reducing tumor growth. Compound 3102 at 30 mg/kg given intravenously was similarly effective in controlling tumor growth. One treatment related death was observed for the compound 3102 70 mg/kg dose, and one non-treatment related death occurred in the controls. Paclitaxel and Taxotere® were as effective or slightly less effective in controlling tumor growth compared to the lower doses of compound 3102 administered both orally and intravenously (Table 22). In addition, there were two non-treatment related deaths in the Taxotere® 30 mg/kg and one non-treatment related and one treatment related death in the Taxotere® 25 mg/kg group. The results of this study show that oral and iv administered Compound 3102 is effective in the treatment of SW480 colon tumors in mice.

TABLE 22
Treatment Response Summary for the SW480-e11 Study

| | | | | | | | Max. % | | |
|---|---|---|---|---|---|---|---|---|---|
| | | | | | | | BW | | |
| | | Regimen 1 | | | | MDS to 2.0 g ± | Loss; | # Death^{a} | |
| Grp | n | Agent | mg/kg | Route | Schedule | SEM(n) | Day | TR | NTR |
| 1 | 6 | Vehicle | — | | Q4D × 4 | 21.7 ± 3.8 (10) | — | 0 | 1 |
| 3 | 6 | Paclitaxel | 30 | IV | QOD × 5 | 26.2 ± 3.0 | −7.5%; | 0 | 0 |
| | | | | | | | Day 12 | | |
| 4 | 6 | Taxotere | 30 | IV | Q7D × 3 | 29.0 ± 7.8 | −24.6%; | 0 | 2 |
| | | | | | | | Day 26 | | |
| 5 | 6 | Taxotere | 25 | IV | Q7D × 3 | 32.4 ± 4.1 | −24.7%; | 1 | 1 |
| | | | | | | | Day 19 | | |
| 6 | 6 | 3102 | 90 | PO | Q4D × 4 | 40.7 ± 1.3 | −23.6%; | 0 | 0 |
| | | | | | | | Day 15 | | |
| 7 | 6 | 3102 | 70 | PO | Q4D × 4 | 29.0 ± 3.8 | −18.1%; | 0 | 1 |
| | | | | | | | Day 19 | | |
| 8 | 6 | 3102 | 50 | PO | Q4D × 4 | 32.3 ± 3.1 | −19.3%; | 0 | 0 |
| | | | | | | | Day 15 | | |
| 12 | 6 | 3102 | 30 | IV | Q4D × 4 | 32.6 ± 4.3 | −20.1%; | 0 | 0 |
| | | | | | | | Day 22 | | |

^{a}# Death: TR (Treatment Related); NTR (Non-Treatment Related)

### EXAMPLE 19

### Study 786-0 e89: Compound 3102, Oral and Intravenous, Multi-Dose, Q4Dx4 with Paclitaxel and Taxotere® as Comparators

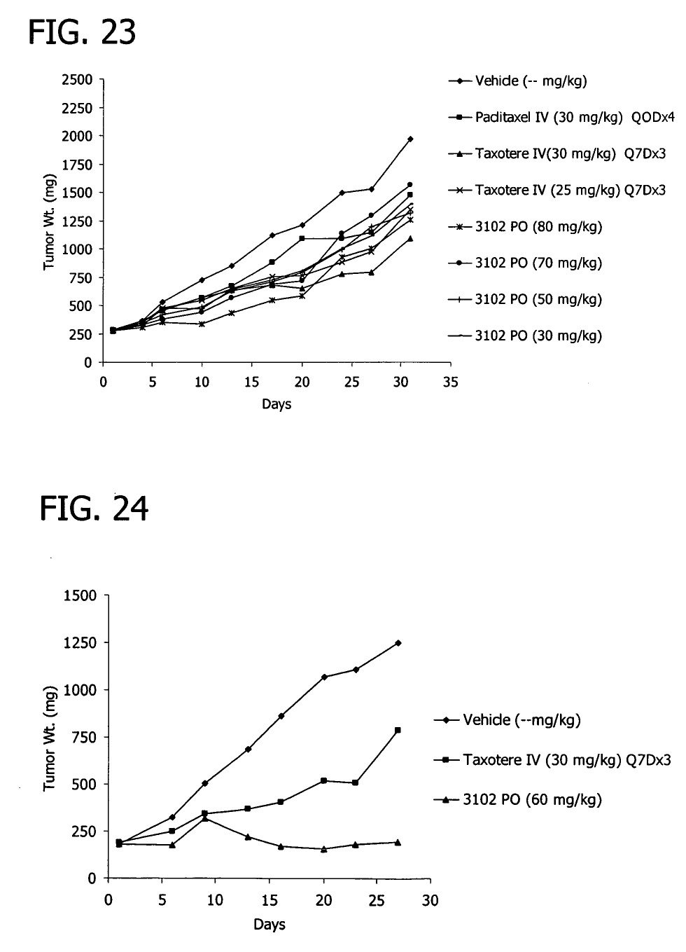

The 786-0 human renal carcinoma was used to evaluate the antitumor activities of orally and intravenously administered compound 3102 using a Q4Dx4 multi-dose schedule. Paclitaxel and Taxotere® were also evaluated in this model at their optimum dose, route (IV) and schedule. The results of this study are presented in FIG. 23 and Tables 23 and 24. FIG. 23 and Table 23 show that both the oral and intravenous administration of compound 3102 resulted in a moderate slowing of the growth of 786-0 tumors in nude mice as indicated by their respective MDS values which were slightly higher compared to control. Paclitaxel and Taxotere® had similar effects. Table 24 is a statistical analyses of the group differences as they relate to tumor growth. The data show that both the high dose (80 mg/kg) orally administered 3102 and the 30 mg/kg intravenous 3102 treatment groups were able to significantly slow the growth of 786-0 tumors in nude mice, compared to the vehicle control (groups are significantly different). Taxotere®, at both dosage levels (30 mg/kg and 25 mg/kg) was also able to slow 786-0 tumor growth compared to the vehicle control (groups are significantly different). However, paclitaxel treatment did not appear to significantly slow the growth of tumors compared to control. These results show that orally administered, compound 3102, on a Q4Dx4 schedule is effective in slowing the growth of 786-0 renal tumors in nude mice.

TABLE 23
Treatment Response Summary for the 786-0-e09 Study

| | | | | | | | Max. % | | |
|---|---|---|---|---|---|---|---|---|---|
| | | | | | | | BW | | |
| | | Regimen 1 | | | | MDS to 2.0 g ± | Loss; | # Death^{a} | |
| Grp | n | Agent | mg/kg | Route | Schedule | SEM(n) | Day | TR | NTR |
| 1 | 6 | 5% EC | — | | | 37.7 ± 2.8 (10) | −4.6%; | 0 | 0 |
| | | in Saline | | | | | Day 24 | | |
| 3 | 6 | Paclitaxel | 30 | IV | QOD × 5 | 40.0 ± 6.6 | −7.6%; | 0 | 0 |
| | | | | | | | Day 13 | | |
| 4 | 6 | Taxotere | 30 | IV | Q7D × 3 | 52.1 ± 4.7 | −23.3%; | 0 | 2 |
| | | | | | | | Day 27 | | |
| 5 | 6 | Taxotere | 25 | IV | Q7D × 3 | 49.3 ± 4.3 | −13.5%; | 0 | 0 |
| | | | | | | | Day 24 | | |
| 6 | 6 | 3102 | 80 | PO | Q4D × 4 | 47.7 ± 2.1 | −7.6%; | 0 | 0 |
| | | | | | | | Day 17 | | |
| 7 | 6 | 3102 | 70 | PO | Q4D × 4 | 40.5 ± 2.0 | −5.3%; | 0 | 0 |
| | | | | | | | Day 10 | | |
| 8 | 6 | 3102 | 50 | PO | Q4D × 4 | 42.8 ± 2.2 | — | 0 | 0 |
| 12 | 6 | 3102 | 30 | IV | Q4D × 4 | 50.8 ± 3.8 | −12.4%; | 0 | 0 |
| | | | | | | | Day 17 | | |

^{a}# Death: TR (Treatment Related); NTR (Non-Treatment Related)

TABLE 24
Statistical Analysis

| Compared Groups | 5% EC | 5% EC | 5% EC | 5% EC | 5% EC | 5% EC | 5% EC |
|---|---|---|---|---|---|---|---|
| | in Saline; | in Saline; | in Saline; | in Saline; | in Saline; | in Saline; | in Saline; |
| | PO; | PO; | PO; | PO; | PO; | PO; | PO; |
| | Q4D × 4 | Q4D × 4 | Q4D × 4 | Q4D × 4 | Q4D × 4 | Q4D × 4 | Q4D × 4 |
| | — | — | — | — | — | — | — |
| | Paclitaxel; | Docetaxel; | Docetaxel; | 3102; | 3102; | 3102; | 3102; |
| | IV; | IV; | IV; | PO | PO | PO | IV |
| | QOD × 5 | QWK × 3 | QWK × 3 | Q4D × 4 | Q4D × 4 | Q4D × 4 | Q4D × 4 |
| | 30 mg/kg | 30 mg/kg | 25 mg/kg | 80 mg/kg | 70 mg/kg | 50 mg/kg | 30 mg/kg |
| Logrank Test | | | | | | | |
| Chi square | 3.414 | 5.271 | 4.859 | 5.234 | 0.2502 | 1.972 | 5.459 |
| df | 1 | 1 | 1 | 1 | 1 | 1 | 1 |
| P value | 0.0646 | 0.0217 | 0.0275 | 0.0221 | 0.6169 | 0.1602 | 0.0195 |
| P value summary | ns | * | * | * | ns | ns | * |
| Are the survival | No | Yes | Yes | Yes | No | No | Yes |
| curves sig | | | | | | | |
| different? | | | | | | | |
| Median survival | | | | | | | |
| Column A | 37.1 | 37.1 | 37.1 | 37.1 | 37.1 | 37.1 | 37.1 |
| Column B | 51.15 | 52 | 52.7 | 48 | 38.8 | 45.05 | 54.65 |
| Ratio | 0.7253 | 0.7135 | 0.704 | 0.7729 | 0.9562 | 0.8235 | 0.6789 |
| 95% CI of ratio | 0.4431 to | 0.4313 to | 0.3815 to | 0.4504 to | 0.6337 to | 0.5183 to | 0.3564 to |
| | 1.008 | 0.9957 | 1.027 | 1.095 | 1.279 | 1.129 | 1.001 |
| Hazard Ratio Ratio | 2.82 | 3.39 | 2.967 | 3.013 | 1.297 | 2.176 | 3.164 |
| 95% of CI ratio | 0.9212 to | 1.301 to | 1.198 to | 1.283 to | 0.3899 to | 0.6885 to | 1.326 to |
| | 16.17 | 27.99 | 21.88 | 25.25 | 4.892 | 9.588 | 25.02 |

### EXAMPLE 20

### Study MSTO 61604: Compound 3102, Oral, Multi-dose, Q4Dx4 with Taxotere® as Comparator

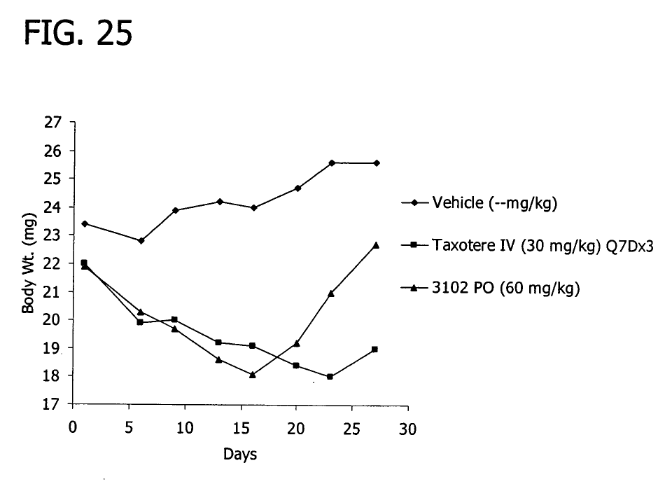

Compound 3102 was evaluated for antitumor activity in the MSTO-211H human mesothelioma mouse xenograft model. Compound 3102 was administered orally on a Q4Dx4 schedule at a dose of 60 mg/kg. Taxotere® was used as a comparator and was administered intravenously at a dose of 30 mg/kg on a Q7Dx3 schedule. The results are presented in FIGS. 24 and 25. Tumors in the vehicle control group reached a maximum tumor wt. of 1250 mg by day 27. Compound 3102 was highly effective in slowing MSTO-211H tumor growth and reducing tumor size and weight to below that of the original implant. Taxotere® was only moderately effective in slowing tumor growth, and tumors grew rapidly following the last dose of Taxotere®. Body weight changes in the compound 3102 and Taxotere® groups were similar for the first 15 days, however, the compound 3102 group recovered weight more rapidly than the Taxotere® group (FIG. 25). These results show that multi-dosed, orally administered compound 3102 is superior to intravenous Taxotere®, in slowing tumor growth and appears to be less toxic as indicated by a more rapid recovery of body weight.

What is claimed is:

1. A method of inhibiting paclitaxel or docetaxel resistant tumor growth in mammals, said method comprising administering a therapeutically effective amount of a pharmaceutical composition comprising a taxane having the formula 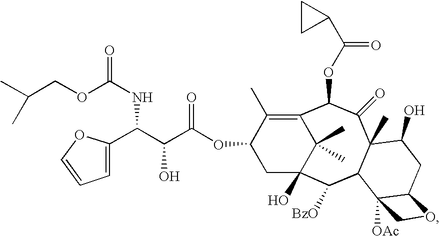
or a pharmaceutically acceptable salt thereof, and at least one pharmaceutically acceptable carrier.

2. The method of claim 1 wherein the tumor is resistant to paclitaxel.

3. The method of claim 1 wherein the tumor is breast, lung, pancreas, colon, ovarian, or prostate carcinoma.

4. The method of any of claims 1-3 wherein the tumor is colon or ovarian carcinoma.

5. The method of any of claims 1-3 wherein the tumor is breast carcinoma.

6. The method of any of claims 1-3 wherein the tumor is HCT116 colon carcinoma, HT-29 colon carcinoma, SW480 colon carcinoma, DLD-1 colon carcinoma, PANC-1 pancreatic adenocarcinoma, PC-3 prostate carcinoma, LNCaP prostate carcinoma, IA9 ovarian carcinoma, IA9-PTX10 ovarian carcinoma, IA9-PTX22 ovarian carcinoma, A375 melanoma, 786-0 renal carcinoma, or MSTO-211H methothelioma.

7. The method of any of claims 1-3 wherein the tumor is HCT116 colon carcinoma, HT-29 colon carcinoma, DLD-1 colon carcinoma, PANC-1 pancreatic adenocarcinoma, PC-3 prostate carcinoma, LNCaP prostate carcinoma, IA9 ovarian carcinoma, IA9-PTX10 ovarian carcinoma, or IA9-PTX22 ovarian carcinoma.

8. The method of any of claims 1-3 wherein the tumor is VM46 human colon carcinoma, DLD-1 human colon carcinoma, 1A9-PTX10 ovarian carcinoma, or 1A9-PTX22 ovarian carcinoma.

9. The method of claim 1 wherein said pharmaceutical composition is administered orally.

10. The method of claim 1 where said pharmaceutical composition is administered parenterally.

11. The method of claim 1 wherein said mammal is pretreated with dexamethasone, diphenhydramine, or other agent that minimizes adverse reactions from the administration of the pharmaceutical composition and the pharmaceutical composition comprises a surfactant.

12. The method of claim 11 wherein said surfactant is polysorbate 80, polyethoxylated caster oil, or a combination thereof.
